(19) United States
(12) Patent Application Publication
(10) Pub. No.: US 2021/0001105 A1
(43) Pub. Date: Jan. 7, 2021

(54) REMOTE ACCESS VASCULAR AND SOFT TISSUE TUNNERLING DILATOR SYSTEM AND METHODS OF USE

(71) Applicant: JT Access, LLC, Fort Lauderdale, FL (US)

(72) Inventor: Gerald Ernst Schmidt, Fort Lauderdale, FL (US)

(21) Appl. No.: 16/911,990

(22) Filed: Jun. 25, 2020

Related U.S. Application Data
(63) Continuation-in-part of application No. 16/888,595, filed on May 29, 2020.
(60) Provisional application No. 62/869,487, filed on Jul. 1, 2019.

(51) Int. Cl. A61M 39/02

(52) U.S. Cl. CPC A61M 39/0247; A61M 2039/0273; A61M 2210/1082; A61M 2039/0279; A61M 2039/0282; A61M 2039/0276

(57) ABSTRACT

Remote access, soft tissue tunneling dilators are used for the placement of central venous catheters of varying sizes into one or more jugular veins, for forming pathways of varying sizes through subcutaneous soft tissue, or for enlarging internal renal access pathways of varying sizes to accommodate access by urological instruments. Dilators comprise a tapered body extending from a proximal end to a distal tip, where both an outer diameter and a material hardness of the tapered body gradually increase distally-to-proximally from the distal tip toward the proximal end of the tapered body. The tapered body may include a plurality of visual indicators disposed upon the body at one or both of a plurality of distally-to-proximally increasing length increments and a plurality of distally-to-proximally increasing diameter increments. Other embodiments are also disclosed.

### REFERENCE TO PENDING PRIOR PATENT APPLICATION

This application is a continuation-in-part of U.S. patent application Ser. No. 16/888,595, filed May 29, 2020 by Gerald Ernst Schmidt for “REMOTE ACCESS VASCULAR AND SOFT TISSUE TUNNELING DILATOR SYSTEMS AND METHODS OF USE,” which claims the benefit under 35 U.S.C. 119 (e) of U.S. Provisional Patent Application No. 62/869,487, filed Jul. 1, 2019 by Gerald Ernst Schmidt and Scott W. Peterson for “REMOTE ACCESS SOFT TISSUE TUNNELING DILATOR FOR PLACEMENT OF CENTRAL VENOUS CATHETERS INTO THE JUGULAR VEINS,” both of which patent applications are hereby incorporated herein by reference.

### BACKGROUND

Percutaneous techniques revolutionized vascular cannulation. In 1953, Dr. Sven Ivar Seldinger, a Swedish radiologist, introduced a medical procedure to obtain safe access to blood vessels and other hollow organs. The Seldinger technique became the basis for the many percutaneous procedures performed today. The desired vessel or cavity is punctured with a sharp hollow needle through which a guidewire is advanced. Such percutaneous procedures essentially eliminated the need for open cutdown procedures and the associated wound-related morbidity. However, initial percutaneous techniques left the operating physician or clinician exclusively reliant upon the relationships between surface anatomic landmarks and the underlying deep anatomic structures. While techniques have improved, and clinicians now insert more than five million percutaneous central venous catheters (CVCs) annually, there remains an overall complication rate of 15%. These complications include infection, thrombosis, occlusion, and, in particular, mechanical complications which usually occur during insertion and are intimately related to the anatomic relationships of the central veins.

There are currently multiple different methods for placement of a central venous catheter (CVC). The most common methods are: Peripherally Inserted Central Catheter (PICC), an Implanted Venous Port, an External Non-Tunneled Central Venous Catheter, a Tunneled Central Venous Catheter, and a Femoral Vein catheter.

The internal jugular vein (IJV) has become the preferred access site for central venous cannulation because of demonstrated reduced complication rates, including reduced rates of thrombosis, pneumothorax, and avoidance of catheter “pinch-off” syndrome. Advantages of accessing the IJV include a superficial location, easy ultrasonic visualization, and a straight course to the superior vena cava (from the right). Internal jugular cannulation avoids the subclavian “pinch-off syndrome.” Furthermore, for renal failure patients, IJV cannulation avoids potential subclavian vein stenosis which would preclude use of the extremity for hemodialysis access via arteriovenous shunt/fistula. There are three percutaneous approaches to the IJV: anterior, central, and posterior.

Most tunneled CVCs that are now placed utilize a two “stick” (incision) approach. This involves an incision to access the IJV with placement of a peel away sheath and a second incision laterally to form a subcutaneous tunnel. The CVC is then placed through the subcutaneous tunnel and looped back through the peel away sheath into the superior vena cava (SVC). In this procedure, the tunnel is made in the soft tissues anterior to the sternocleidomastoid muscle (SCM).

A single stick placement was first described by Bradley Glenn MD in the Journal of Vascular Interventional Radiology in 2007. He used ultrasound to guide the needle for puncture of the IJV and then used a straight dilator with a hand-made or hand-bent curve to tunnel anteriorly to the SCM through the soft tissues.

In addition, many of today's advanced procedures require the additional use of serial dilators of increasing diameter, as well as sheaths, to form the subcutaneous tract or pathway that will receive the catheter. Existing systems utilize straight vascular dilators for the placement of CVCs. In this regard, conventional CVC kits generally comprise at least four separate components, namely, a syringe coupled to a needle having a longitudinal lumen, a guide wire, multiple progressively sized straight dilators, and a CVC. Multiple straight dilators are advanced over the guide wire to dilate tissue and vein around the guide wire to facilitate the CVC and are withdrawn prior to the CVC being placed in the access pathway created by the dilator.

In addition to vascular dilation of blood vessels, dilators are commonly used for soft tissue dilation in multiple interventional procedures including, for example, access to the kidneys, stomach, liver, peritoneal cavity, and for abscess drainage and the dilation of urethral strictures. Generally, the vascular, renal, and fascial dilation applications involve exchanging multiple dilators of progressively larger diameters until the final subcutaneous tract is sufficiently large for the requisite application. These progressively larger dilators are typically provided in a kit 20 including a catheter, multiple dilators 22 in an increasing spectrum of sizes (e.g., 8 FR-30 FR), and a variety of corresponding sheaths 24, as shown in prior art FIG. 1. This approach requires multiple-component dilator systems at greater cost, and also mandates less-efficient, multiple-step methods of use for forming subcutaneous tracts or pathways within the body.

Moreover, there are occasions when because of radiation, postoperative scarring, lymphadenopathy, or other etiologies, there are blockages or strictures in certain structures including the arteries, veins, ureters, intestines, bile ducts and many other structures which lead to significant health problems. Previously this has been addressed by dilating the structure with an angioplasty balloon catheter. However, angioplasty alone has many limitations including whether access can be obtained across the blockage, whether the angioplasty balloon can make certain bends to reach the site of pathology, as well as other limitations.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides a remote access, tunneling dilator for forming a subcutaneous tract within a human body. The remote access, tunneling dilator may include a tapered body extending from a proximal end to a distal tip with a proximal body section and a straight tapered section disposed proximally-to-distally therebetween, wherein: (1) the straight tapered section extends along a straight tapered length having an outer diameter that gradually increases distally-to-proximally from a distal body diameter to a maximum body diameter; and (2) the proximal body section extends along a proximal body length having the maximum body diameter.

Another embodiment provides a continuously tapered dilator for enlarging an internal pathway within a human body. The continuously tapered dilator may include a body extending from a proximal end to a distal tip with at least a proximal body section, a straight tapered section, and a distal end section disposed proximally-to-distally therebetween, wherein the body has a tapered diameter that increases distally-to-proximally from a minimum diameter at the distal end section to a maximum diameter at the proximal body section.

Yet another embodiment provides a remote access, tunneling dilator for enlarging a subcutaneous tract within a human body. The remote access, tunneling dilator may include a body extending from a proximal end to a distal tip, the body having: (1) a tapered outer diameter that increases distally-to-proximally from a minimum diameter at the distal tip to a maximum diameter at the proximal end; (2) a variable material hardness in which the distal tip has a first material hardness, the proximal end has a second material hardness, and the second material hardness is greater than the first material hardness; (3) a plurality of visual indicators disposed upon the body at one or both of a plurality of distally-to-proximally increasing length increments and a plurality of distally-to-proximally increasing diameter increments; and (4) a through hole extending from the proximal end to the distal end, the through hole configured to receive a standard guide wire.

Still another embodiment provides a method of dilating a vascular system for remotely placing a catheter via a jugular vein of a patient. The method may include the steps of (1) providing a tapered vascular dilator having a tapered body extending from a proximal end to a distal tip and having a proximal body section and a straight tapered section disposed proximally-to-distally therebetween, wherein: (a) the straight tapered section extends along a straight tapered length having an outer diameter that slopes distally-to-proximally from a minimum distal diameter to a maximum proximal diameter; and (b) the proximal body section extends along a proximal body length having the maximum proximal diameter; (2) using an introducer needle, puncturing an internal jugular vein (IJV) from a remote access point posterior to a sternocleidomastoid muscle (SCM) of the patient; (3) advancing, distally-to-proximally, the tapered vascular dilator over a standard guide wire from the remote access point, through a plurality of soft tissues surrounding the guide wire, and into an inferior vena cava (IVC), thereby dilating an access pathway from the remote access point to the IVC in not more than one dilation step, the access pathway having a diameter sized to receive the catheter to facilitate catheterization; (4) removing the tapered vascular dilator; and (5) placing the catheter over the guide wire and through the access pathway created by the tapered vascular dilator into the IVC.

Another embodiment provides a method of dilating a subcutaneous access pathway to a target area within a body of a patient. The method may comprise forming, using not more than one tapered fascial dilator, an access pathway from an access point on a skin surface of the patient to the target area within the body. The forming the access pathway comprises advancing the tapered fascial dilator, distally-to-proximally, over a guide wire from the access point, through a plurality of soft tissues surrounding the guide wire, and into the target area within the body, the access pathway having a diameter sized to receive a medical implement.

A further embodiment provides a method of forming an access pathway to a target area of a kidney of a patient using a renal access dilator comprising a tapered body extending from a proximal end to a distal tip, the body having a tapered outer diameter that increases distally-to-proximally from a minimum diameter at the distal tip to a maximum diameter at the proximal end. The method may include the steps of (1) advancing an introducer needle from an access point on a posterior flank of the patient into the target area of the kidney; (2) advancing a guide wire through the introducer needle into the target area of the kidney; (3) advancing the renal access dilator, distally-to-proximally, over the guide wire from the access point, through a plurality of soft tissues surrounding the guide wire, and into the target area of the kidney, thereby dilating the plurality of the soft tissues along the guide wire from a first state in which the plurality of the soft tissues confront the guide wire to a second state in which the plurality of the soft tissues form a renal access pathway having a diameter sized to receive a select urology instrument; (4) withdrawing the renal access dilator; and (5) placing a sheath over the guide wire such that the sheath and the guide wire together maintain the renal access pathway for the select urology instrument from the access point into the target area of the kidney.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 provides a perspective view of a prior art dilator kit including a catheter, multiple dilators in an increasing spectrum of diameters, and a variety of corresponding sheaths;

FIGS. 2A-2B provide schematics detailing the veins of the neck and the thoracic and abdominal regions of the human body;

FIGS. 3A-3D illustrate respective top plan, side, partial, and partial-cross-section views of one embodiment of a remote access, tunneling dilator for the placement of central venous catheters of varying sizes into the jugular veins;

FIGS. 4A-4B illustrate respective perspective and section views of a distal tip of the dilator of FIGS. 3A-3D;

FIG. 5 illustrates a top plan view of another embodiment of a remote access, tunneling dilator for the placement of central venous catheters of varying sizes into the jugular veins;

FIG. 6 illustrates a top plan view of another embodiment of a remote access, tunneling dilator for the placement of central venous catheters of varying sizes into the jugular veins;

FIG. 7 illustrates a top plan view of another embodiment of a remote access, tunneling dilator for the placement of central venous catheters of varying sizes into the jugular veins;

FIG. 8 illustrates a top plan view of another embodiment of a remote access, tunneling dilator for the placement of central venous catheters of varying sizes into the jugular veins;

FIG. 9 illustrates a top plan view of another embodiment of a remote access, tunneling dilator for the placement of central venous catheters of varying sizes into the jugular veins;

FIG. 10 provides a flowchart depicting an exemplary method of using embodiments of the remote access, tunneling dilators of FIGS. 3A-3D, 4A-4B, and 5-9 to remotely place catheters of varying sizes;

FIG. 11 illustrates a top plan view of one embodiment of a fascial dilator for forming pathways of varying sizes through subcutaneous soft tissue;

FIG. 12 illustrates a top plan view of another embodiment of a fascial dilator for forming pathways of varying sizes through subcutaneous soft tissue;

FIG. 13 illustrates a top plan view of another embodiment of a fascial dilator for forming pathways of varying sizes through subcutaneous soft tissue;

FIG. 14 illustrates a top plan view of one embodiment of a renal access dilator for forming internal renal access pathways of varying sizes to accommodate access by urological instruments;

FIG. 15 illustrates a top plan view of another embodiment of a renal access dilator for forming internal renal access pathways of varying sizes to accommodate access by urological instruments;

FIGS. 16A-16B illustrate respective anterior cross-sectional and lateral cross-sectional views of a human peritoneal cavity having a pelvic abscess;

FIG. 16C illustrates a lateral cross-sectional view of a human peritoneal cavity undergoing peritoneal drainage and dialysis;

FIG. 17 provides a flowchart depicting an exemplary method of using embodiments of the remote access, tunneling facial dilators of FIGS. 11-13 for dilating a pathway for placement of a peritoneal catheter for peritoneal drainage and dialysis of a pelvic abscess;

FIGS. 18A-18B illustrate a posterior view of a right kidney as positioned within a patient's posterior flank and an anterior cross-sectional view of the right kidney, respectively; and

FIG. 19 provides a flowchart depicting an exemplary renal access dilation method using embodiments of the remote access, tapered renal access dilators of FIGS. 14-15.

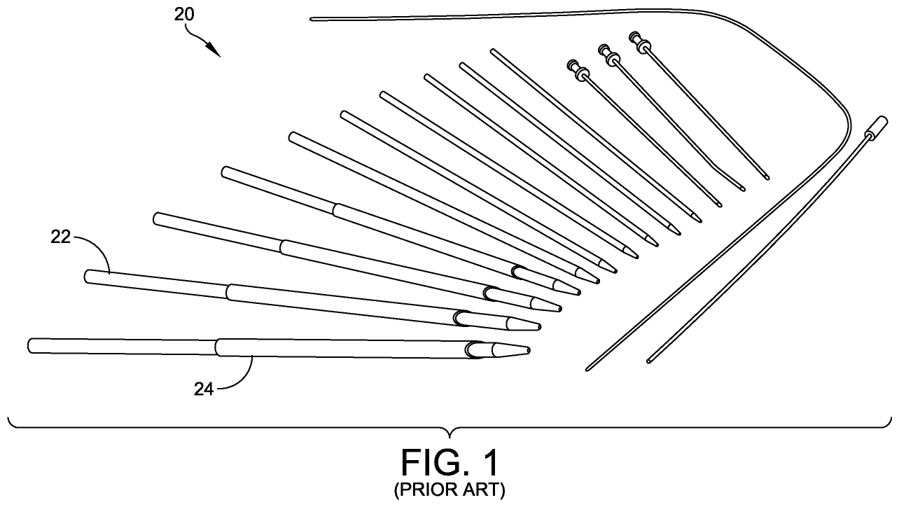

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

The disclosure discusses systems and methods of use pertaining to dilation systems and related methods of vascular and soft tissue dilation. The systems and methods are for gaining access to portions of a patient's body by a clinician, for example, to place central venous catheters (CVCs) into the jugular veins, to obtain percutaneous access to the kidney by a urologist or a radiologist for approach by urological instruments and for renal percutaneous procedures, for the placement of drainage catheters or gastrostomy feeding tubes, and/or for dilating any strictures and/or obstructions inside the soft tissues or vascular systems of the body.

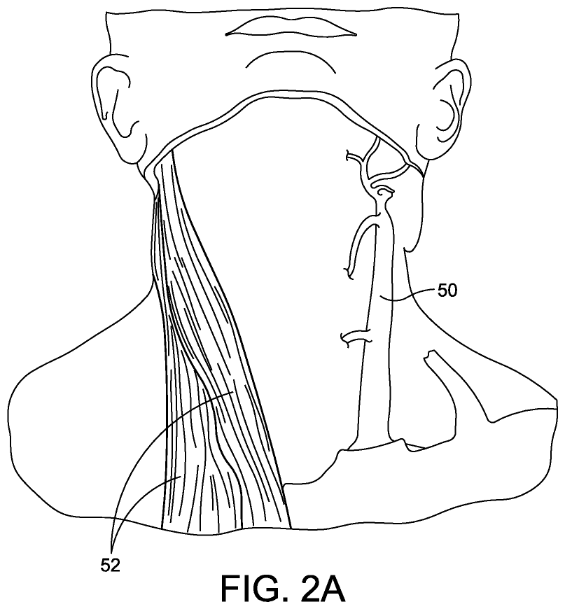

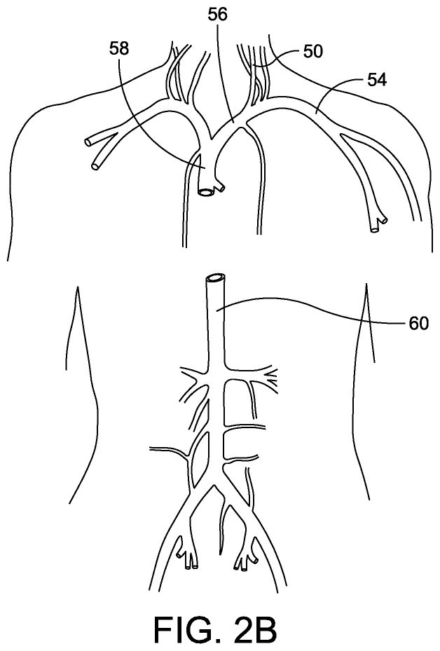

In particular, the present invention relates to dilation systems for dilating a tract or pathway opening to a desired size, from very small in diameter (e.g., 5 FR) to very large in diameter (e.g., 30 FR), and maintaining that opening with a single dilator rather than multiple progressive dilators. In this regard, both vascular and soft tissue dilation may be achieved in a manner that is safe and effective, and that enables the most advantageous route through the body without requiring the exchange of multiple dilators that are graduated in diameter size. To aid explanation, FIGS. 2A-2B schematically illustrate the human anterior thoracic wall and the chest and renal cavities, respectively, for describing placement of central venous catheters (CVCs) into the jugular veins, as well as for describing soft tissue dilation applications including renal access and fascial dilation applications including gastrostomy feeding tubes, abscess drainage, peritoneal drains or external biliary drainage.

I. Vascular Dilation

Various embodiments of the systems and methods described in this section relate to remote access, tunneling dilators for vascular applications. Some embodiments provide tunneling dilators for the placement of CVCs into the jugular veins, configured for use with a straight or a pre-bent needle and a standard stiff guide wire in creating an access pathway through the skin and a jugular vein such as, for example, the internal jugular vein (IJV) 50 (FIGS. 2A-2B). In one embodiment, the access pathway through the IJV 50 may be made via an approach that is posterior to the sternocleidomastoid muscle (SCM) 52 (FIG. 2A).

Embodiments of the remote access, soft tissue tunneling dilator provide a number of advantages over existing dilators and provide a safe and effective mechanism for achieving remote access via a single stick, or single incision, placement of a CVC into the IJV 50 using an approach that is posterior to the SCM 52, without the use of a sheath.

In some embodiments, the remote access, tunneling dilator features a permanent curve configured to navigate subcutaneous curves in the body as the dilator descends through the IJV 50 and enters the left subclavian vein 54 and then the innominate (or brachiocephalic) vein 56 (FIGS. 2A-2B).

Embodiments of the remote access, tunneling dilator also feature a continuously tapered exterior having a diameter with a progressive French size that eliminates the need for utilizing multiple progressively sized dilators to accommodate different sizes of CVC. Currently, up to ten separate dilators must be progressively exchanged to accommodate the largest catheters (e.g., Hemodialysis catheters 14 Fr and Angio-Vac 24 Fr). In addition to excessive time and expense, these dilator exchanges cause bleeding at the puncture site and can increase chances of infection.

In addition, the continuously tapered configuration allows embodiments of the dilator to be advanced through vascular narrowings, or strictures, without the use of an angioplasty balloon catheter, which saves both procedure time and the added expense of a dilator balloon kit. Because the continuous taper enables embodiments of the remote access, tunneling dilator to be advanced through vascular strictures, the clinician may gain access through a vein that would otherwise be unavailable, thereby forcing clinician to choose a less advantageous route. Embodiments of the tapered dilator allow easy treatment of and therapeutic access through these strictures. This reduces procedural time and also allows treatment of some conditions which were not previously considered for minimally invasive surgical techniques.

Additional embodiments of the tunneling dilator disclosed herein feature a varying material hardness (shore durometer) over a length of the dilator. That is, a distal section of the dilator may be formed from a softer material, allowing the dilator to be more easily maneuvered as it progresses around and posterior to the SCM 52 or other subcutaneous curves without risk of material folding that may kink the guide wire.

Embodiments of the remote-access, tunneling dilator may also feature visual indicators marking the dilator diameter and/or the dilator length at defined increments along the dilator body, providing a convenient mechanism by which a clinician may gauge the diameter of the tract being formed within the body and/or the requisite length of the catheter necessary to reach a target position within the body.

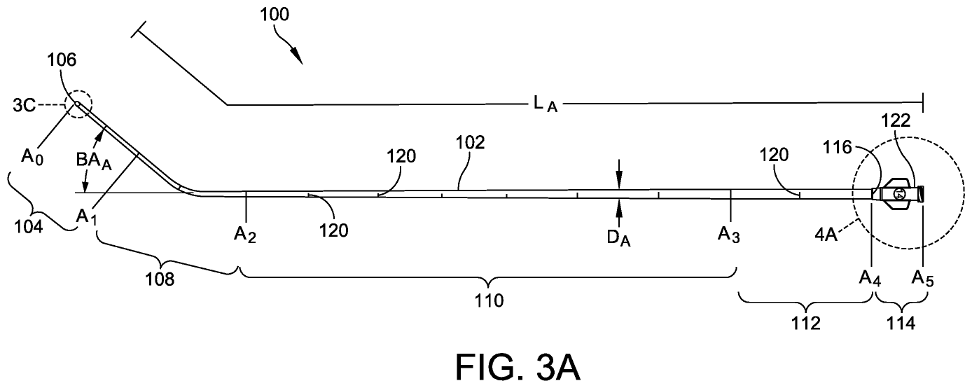

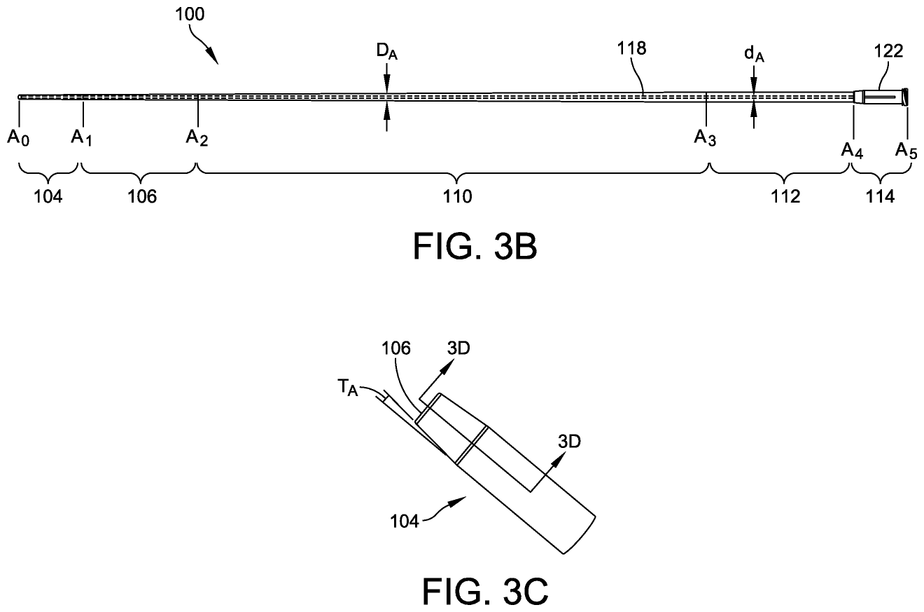

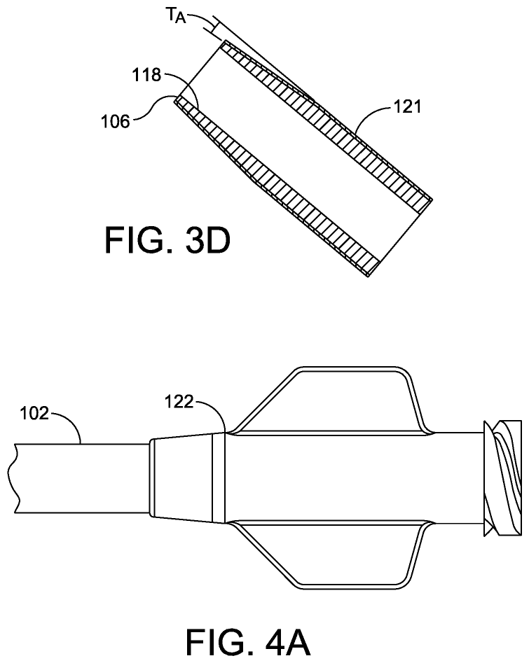

Turning to exemplary embodiments, FIGS. 3A-3D illustrate respective top plan, side, partial, and partial cross-sectional views of one embodiment of a remote access, curved tunneling dilator 100 for use in placing CVCs into the jugular veins. In this embodiment, the curved dilator 100 has a total length, L_{A}, of 43.5 cm separated into five zones extending between Markers A₀-A₁, A₁-A₂, A₂-A₃, A₃-A₄, and A₄-A₅, including a dilator body 102 having a distal end section 104 extending 4 cm from a distal tip 106 at Marker A₀ to Marker A₁, a curved section 108 extending 6 cm from Markers A₁-A₂, a straight tapered section 110 extending 24 cm between Markers A₂-A₃, and a proximal body section 112 extending 7 cm between Markers A₃-A₄. In this embodiment, the dilator 100 further includes a connector end section 114 disposed adjacent to a proximal end 116 of the dilator body 102, extending 2.5 cm from Marker A₄ to a terminal proximal end of the dilator 100 at Marker A₅.

In one embodiment, a longitudinal through hole 118 may extend through an entirety of the length, L_{A}, of the dilator 100. The longitudinal through hole 118 may have an inner diameter, d_{A}, configured to accommodate a standard guide wire (e.g., 0.035 inch diameter, 0.038 inch diameter) (not shown). In addition, the dilator body 102 may include a hydrophilic outer coating 121 over an entirety of its length for added lubricity to more easily slip through subcutaneous tissues.

In this embodiment, the body 102 of the dilator 100 may form a permanent curve or angle in the curved section 108 extending between Markers A₁-A₂, as shown in FIG. 3A. In this embodiment, the permanent curve may form a body angle, BAA, of approximately 40 degrees relative to the straight tapered section 110 extending between Markers A₂-A₃. Notably, the permanent curve, or body angle, BAA, may form any appropriate angle including, for example, an angle of 5 degrees, 45 degrees, or 90 degrees, depending on the intended dilation application.

The permanent curve enables embodiments of the dilator 100 to better navigate subcutaneous curves and bodily features to establish remote access via the jugular veins. This is most pronounced when establishing remote access via the left IJV 50 (FIG. 2A), as is necessary in about 10% of CVC placements. A posterior-lateral approach to the left IJV 50 involves the dilator navigating two curves as the dilator descends from the IJV 50 and enters the left subclavian vein 54, and then the innominate/brachiocephalic vein 56 (FIGS. 2A-2B). Due to the difficulty of navigating these curves, the traditional approach uses a non-curved sheath, which may lead to venous perforation and catastrophic consequences.

In addition to the permanent curve or body angle, BAA, the body 102 of the dilator 100 may feature a tapered configuration having a tapered outer diameter, D_{A}, that progresses in diameter size distally-to-proximally from the distal tip 106 to the proximal end 116 of the body 102, beginning, in this embodiment, with an outer diameter of 5 FR on the French catheter scale at the distal end section 104 extending between Markers A₀-A₁, increasing to 6 FR through the curved section 108 extending between Markers A₁-A₂, then progressively increasing by 1 FR every 3 cm through the straight tapered section 110 from 6 FR to 14 FR between Markers A₂-A₃, reaching and maintaining a maximum diameter of 14 FR at the proximal body section 112 extending between Markers A₃-A₄, as detailed in FIGS. 3A-3B. In some embodiments, the distal tip 106 of the dilator body 102 may additionally be tapered by a tip angle, TA, of approximately 5 degrees, as shown in FIGS. 3C-3D, for additional ease of insertion and manipulation.

Visual indicators 120 may be disposed upon the body 102 of the dilator 100 at each increasing diameter progression to enable a clinician to determine the outer diameter size entering the body, and, in turn, the size of the remote access pathway that will be created by the dilator 100. Additionally or alternatively, the visual indicators 120 may be disposed on the body at defined length increments along the total length, L_{A}, of the dilator 100, enabling the clinician to quickly determine the length of CVC necessary for the particular application based upon the visual indicator 120 aligned at the entry point on the patient's skin when the distal tip 106 of the dilator 100 reaches its subcutaneous target within the patient's body (e.g., within the superior vena cava (SVC) 58 right above the heart). In the prior art, this CVC length determination is made by marking the guide wire when its distal end is seen at the ideal location in the SVC. The guide wire is then removed to measure its length before reinserting the guide wire, extracting the final (i.e., largest) dilator from the body, and placing the CVC. This method requires an extra step and introduces error into the length determination. The visual indicators 120 (e.g., stripes or tick marks) may be pad printed or laser marked on the outer (i.e., curved) side of the dilator body to maximize radiological/fluoroscopic visibility of the indicators 120 for use in directing the dilator 100 through the body during a procedure.

As discussed above, the progressively increasing outer diameter, D_{A}, of the dilator body 102 renders the dilator 100 suitable for the placement of a variety of catheter sizes. Rather than exchanging discrete dilators of increasing diameter and confronting the associated risks discussed above, the clinician may use a long guide wire along with a single remote access, curved tunneling dilator 100 having the progressive French sizing for a posterior approach through, for example, the IJV 50, to the superior vena cava 58, and on to the inferior vena cava 60 (FIGS. 2A-2B).

In one embodiment, the body 102 of the remote access, curved tunneling dilator 100 may also feature a variable material hardness. In this embodiment, the material of the body may increase in material hardness or Shore durometer rating from the distal tip 106 at Marker A₀ toward the proximal end 116 of the body 102 at Marker A₄. For example, the distal end section 104 extending between Markers A₀-A₁ may be formed of the softest, least resistant material having a hardness of Shore 25D, forming a soft dilator tip. The curved or angled section 108 between Markers A₁-A₂ may increase in hardness to a hardness of Shore 50D. Progressing distally-to-proximally, the straight tapered section 110 extending between Markers A₂-A₃ and the proximal body section 112 extending between Markers A₃-A₄ may each have a hardness of Shore 60D.

The soft dilator tip, or the distal end section 104, enables flex in the dilator tip as it traverses bodily tissues. This prevents both kinking of the guide wire and puncturing of the venous sidewalls as the remote access, tunneling dilator 100 navigates curves within the body. Notably, the increasing material hardness or Shore durometer ratings of the dilator body 102 may vary as appropriate to accommodate the intended use of the dilator 100. For example, varying curves in the body, discussed above, may result in different hardness ratings along the length of the dilator body 102 to ensure structural integrity of the dilator.

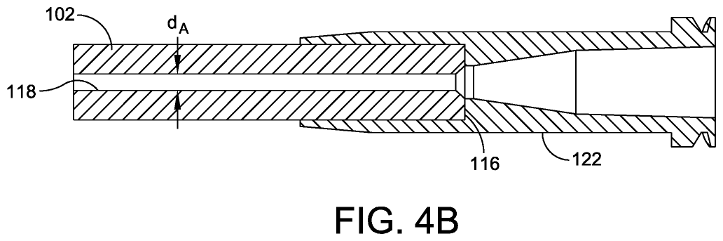

The connector end section 114 of the dilator 100 may comprise a hub 122 such as, for example, a standard female tapered Luer lock hub formed of polycarbonate, as detailed in FIGS. 4A-4B, to allow attachment of other Luer fitting devices to the dilator 100 for use in, for example, the injection of contrast dye. In some embodiments, the longitudinal through hole 118 through the dilator body 102 may be tapered outward at the proximal end 116 of the body 102 to provide a lead-in from the hub 122 to the body 102 for the guide wire.

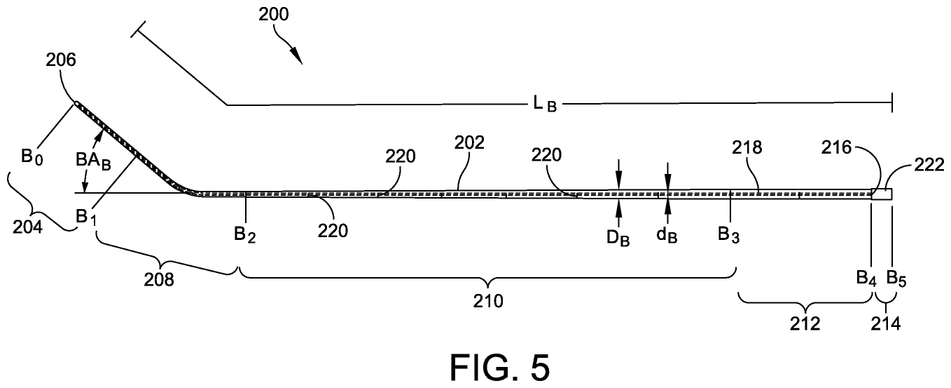

FIG. 5 illustrates a side view of another exemplary embodiment of a remote access, curved tunneling dilator 200 for use in placing CVCs into the jugular veins. The dilator 200 may have similar configuration to the dilator 100, with differing length and taper dimensions and material hardness variations. In this embodiment, the curved dilator 200 has a total length, L_{B}, of 35 cm separated into five zones extending between Markers B₀-B₁, B₁-B₂, B₂-B₃, B₃-B₄, and B₄-B₅, including a dilator body 202 having a distal end section 204 extending 4 cm from a distal tip 206 at Marker B₀ to Marker B₁, a curved section 208 extending 6 cm from Markers B₁-B₂, a straight tapered section 210 extending 14 cm between Markers B₂-B₃, and a proximal body section 212 extending 10 cm between Markers B₃-B₄. The dilator 200 further includes a connector end section 214 disposed adjacent to a proximal end 216 of the dilator body 202, extending 1 cm from Marker B₄ to a terminal proximal end of the dilator 200 at Marker B₅. The connector end section 214 may comprise a 1 cm hub 222 such as, for example, a standard female tapered Luer lock hub to allow attachment of other Luer fitting devices to the dilator 100 for the injection of contrast dye.

In one embodiment, a longitudinal through hole 218 may extend through an entirety of the length, L_{B}, of the dilator 200. The longitudinal through hole 218 may have an inner diameter, d_{B}, configured to accommodate a standard guide wire (e.g., 0.035 inch diameter, 0.038 inch diameter) (not shown). In addition, the dilator body 202 may feature a hydrophilic outer coating over an entirety of its length for added lubricity to more easily slip through subcutaneous tissues.

In this embodiment, the body 202 of the dilator 200 may form a permanent curve or angle in the curved section 208 extending between Markers B₁-B₂. In this embodiment, the permanent curve may form a body angle, BAB, between 40-80 degrees relative to the straight tapered section 210 extending between Markers B₂-B₃. Notably, in some embodiments, the permanent curve, or body angle, BAB, may form any appropriate angle including for example, an angle of 5 degrees, 45 degrees, or 90 degrees, depending on the intended dilation application.

In addition to the permanent curve or body angle, BAB, the body 202 of the dilator 200 may feature a tapered configuration having a tapered outer diameter, D_{B}, that progresses in diameter size distally-to-proximally from the distal tip 206 to the proximal end 216 of the body 202, beginning, in this embodiment, with an outer diameter of 5 FR at the distal end section 204 extending between Markers B₀-B₁, increasing to 6 FR through the curved section 108 extending between Markers B₁-B₂, then progressively increasing by 1 FR every 2 cm through the straight tapered section 210 from 7 FR to 14 FR between Markers B₂-B₃, reaching and maintaining a maximum diameter of 14 FR at the proximal body section 212 extending between Markers B₃-B₄. In some embodiments, the distal tip 206 of the dilator body 202 may additionally be tapered by approximately 5 degrees, in a manner that renders the tip rounded rather than blunt, for additional ease of insertion and manipulation.

Visual indicators 220 may be disposed upon the body 202 of the dilator 200 at each increasing diameter progression to enable a clinician to determine the outer diameter size entering the body, and, in turn, the size of the remote access pathway that will be created by the dilator 200. Additionally or alternatively, the visual indicators 220 may be disposed on the body at defined length increments along the total length, L_{B}, of the dilator 200, enabling the clinician to quickly determine the length of CVC necessary for the particular application based upon the visual indicator 220 aligned with an entry point on the patient's skin when the distal tip 206 of the dilator 200 reaches its subcutaneous target within the patient's body.

In one embodiment, the body 202 of the remote access, curved tunneling dilator 200 may also feature a variable material hardness along the length of the body, L_{B}. For example, in one embodiment the entire 35 cm length, L_{B}, of the body 202 may have a material hardness or Shore durometer rating of 65D. Alternatively, the material of the body 202 may increase in hardness to Shore 80D-90D within the 14 cm straight tapered section 210 between Markers B₂-B₃.

Additional embodiments of the dilator 200 may vary in section length, taper, and material hardness as desired and/or appropriate. For example, in one embodiment, the tapered section 210 may extend 7 cm between Markers B₂-B₃ for a total length, L_{B}, of 28 cm, while distally-to-proximally increasing in diameter by 1 FR every 1 cm from 7 FR to 14 FR, reaching the maximum diameter of 14 FR at Marker B₃. In this configuration, the entire 28 cm length, L_{B}, of the body 202 may have a hardness of shore 65D, or alternatively, the material of the body 202 may increase in hardness to Shore 80D-90D within the 7 cm straight tapered section 210 between Markers B₂-B₃. In each embodiment, however, the hardness may return or remain Shore 65D in the proximal body section 212 between Markers B₃-B₄ to provide stability at the maximum diameter.

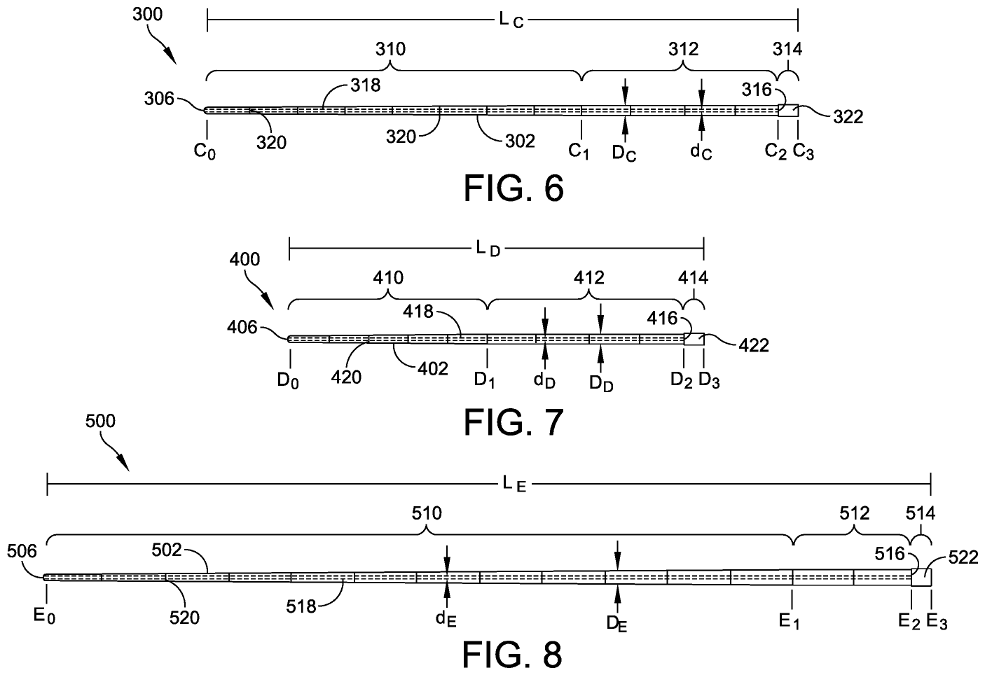

FIGS. 6-9 illustrate side views of various straight, remote access, vascular dilators, each having a different overall length and taper angle. Specifically, FIG. 6 illustrates a side view of one embodiment of a remote access, straight tunneling dilator 300 for use in placing CVCs into the jugular veins. In this embodiment, the straight dilator 300 is shown broken down into 3 zones extending between Markers C₀-C₁, C₁-C₂, and C₂-C₃ for a total length, L_{C}, of 29 cm, including a dilator body 302 having a tapered section 310 extending 18 cm from a rounded distal tip 306 at Marker C₀ to Marker C₁ and a proximal body section 312 extending 10 cm between Markers C₁-C₂. The dilator 300 further includes a connector end section 314 disposed adjacent to a proximal end 316 of the dilator body 302, extending 1 cm from Marker C₂ to a terminal proximal end of the dilator 300 at Marker C₃. The connector end section 314 of the dilator 300 may comprise a hub 322 such as, for example, a standard female tapered Luer lock hub to allow attachment of other Luer fitting devices to the dilator 300 for the purpose of, for example, injecting contrast dye.

In one embodiment, a longitudinal through hole 318 may extend through an entirety of the length of the dilator 300. The longitudinal through hole 318 may have an inner diameter, d_{C}, configured to accommodate a standard guide wire (e.g., 0.035 inch diameter) (not shown). In addition, the dilator body 302 may include a hydrophilic outer coating over an entirety of its length for added lubricity to more easily slip through subcutaneous tissues.

In this embodiment, the body 302 of the dilator 300 may feature a tapered configuration having a tapered outer diameter, D_{C}, that progresses in diameter size distally-to-proximally from the distal tip 306 to the proximal end 316 of the body 302, beginning, in this embodiment, with an outer diameter of 5 FR at the distal tip 306, then progressively increasing through the straight tapered section 310 by 1 FR every 2 cm, from 5 FR to 14 FR, between Markers C₀-C₁, and reaching and maintaining a maximum diameter of 14 FR at the non-tapered proximal body section 312 extending between Markers C₁-C₂, as detailed in FIG. 6.

Visual indicators 320 may be disposed upon the body 302 of the dilator 300 at each increasing diameter progression to enable a clinician to determine the outer diameter size entering the body, and, in turn, the size of the remote access pathway that will be created by the dilator 300. Additionally or alternatively, the visual indicators 320 may be disposed on the body 302 at defined length increments along the total length, L_{C}, of the dilator 300, enabling the clinician to quickly determine the length of CVC necessary for the particular application based upon the visual indicator 320 aligned at the entry point when the distal tip 306 of the dilator 300 reaches its subcutaneous target within the patient's body. The visual indicators 320 (e.g., stripes or tick marks) may be pad printed or laser marked on the outer side of the dilator body to maximize radiological/fluoroscopic visibility of the indicators 320 for use in directing the dilator 300 through the body during a procedure. In addition, the dilator body 302 may feature a hydrophilic outer coating over an entirety of its length for added lubricity to more easily slip through subcutaneous tissues.

FIGS. 7-9 illustrate side views of additional exemplary straight, remote access, vascular dilators 400, 500, and 600. Each of the dilators 400, 500, 600 may be similar in configuration to dilator 300 and includes the same sections and components, discussed above, with varying section lengths and taper slopes to meet the needs of different vascular dilation applications (e.g., pathway lengths, pathway configurations, patient sizes). More specifically and in one embodiment, dilator 400 of FIG. 7 may have a total length, L_{D}, of 20 cm extending between a rounded or angled distal tip 406 and a proximal end of a hub 422. A body 402 of the dilator 400 may feature a tapered configuration having a tapered outer diameter, D_{D}, that progresses in diameter size distally-to-proximally from the distal tip 406 to a proximal end 416 of the body 402, beginning, in this embodiment, with an outer diameter of 5 FR at the distal tip 406 and progressively increasing through a 9 cm straight tapered section 410 by 1 FR every 1 cm, from 5 FR to 14 FR, between Markers D₀-D₁, reaching and maintaining a maximum diameter of 14 FR through a 10 cm proximal body section 412 extending between Markers D₁-D₂. The dilator 400 further includes a connector end section 414 disposed adjacent to the proximal end 416 of the dilator body 402, extending 1 cm from Marker D₂ to a terminal proximal end of the dilator 400 at Marker D₃. The dilator 400 may include a through hole 418 having a diameter, d_{D}, configured to accommodate a standard guide wire (e.g., 0.035 inch) and may include sets of visual indicators 420 disposed at predetermined and progressive length and/or diameter increments along the body 402 to assist the clinician in determining a depth and/or diameter of the resulting tract or passageway formed within the patient's body. In addition, the dilator body 402 may feature a hydrophilic outer coating over an entirety of its length for added lubricity to more easily slip through subcutaneous tissues.

Dilator 500, shown in FIG. 8, may have a total length, L_{E}, of 45 cm extending between a rounded or angled distal tip 506 and a proximal end of a hub 522. A body 502 of the dilator 500 may feature a tapered configuration having a tapered outer diameter, D_{E}, that progresses in diameter size distally-to-proximally from the distal tip 506 to a proximal end 516 of the body 502, beginning, in this embodiment, with an outer diameter of 5 FR at the distal tip 506 and progressively increasing through a 38 cm straight tapered section 510 by 1 FR every 2 cm, from 5 FR to 24 FR, between Markers E₀-E₁, reaching and maintaining a maximum diameter of 24 FR at a 6 cm non-tapered proximal body section 512 extending between Markers E₁-E₂. The dilator 500 may further include a connector end section 514 disposed adjacent to the proximal end 516 of the dilator body 502, extending 1 cm from Marker E₂ to a terminal proximal end of the dilator 500 at Marker E₃. The dilator 500 may include a through hole 518 configured to accommodate a standard guide wire (e.g., 0.038 inch) and may include sets of visual indicators 520 disposed at predetermined and progressive length and/or diameter increments along the body 402 to assist the clinician in determining a depth and/or diameter of the resulting tract or passageway formed within the patient's body. In addition, the dilator body 502 may feature a hydrophilic outer coating over an entirety of its length for added lubricity to more easily slip through subcutaneous tissues.

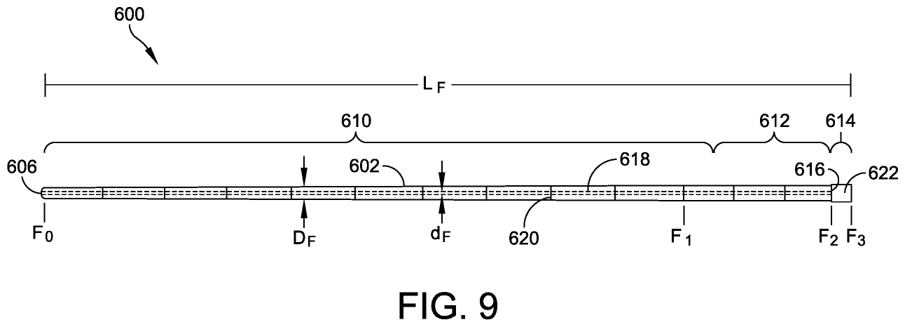

Dilator 600, shown in FIG. 9, may have a total length, L_{F}, of 25 cm extending between a rounded or angled distal tip 606 and a proximal end of a hub 622. A body 602 of the dilator 600 may feature a tapered configuration having a tapered outer diameter, D_{F}, that progresses in diameter size distally-to-proximally from the distal tip 606 to a proximal end 616 of the body 602, beginning, in this embodiment, with an outer diameter of 5 FR at the distal tip 606 and progressively increasing through a 19 cm straight tapered section 610 by 1 FR every 1 cm, from 5 FR to 24 FR, between Markers F₀-F₁, reaching and maintaining a maximum diameter of 24 FR through a 5 cm non-tapered proximal body section 612 extending between Markers F₁-F₂. The dilator 600 may further include a connector end section 614 disposed adjacent to the proximal end 616 of the dilator body 602, extending 1 cm from Marker F₂ to a terminal proximal end of the dilator 600 at Marker F₃. The dilator 600 may also include a through hole 618 configured to accommodate a standard guide wire (e.g., 0.038 inch) and may include sets of visual indicators 620 disposed at predetermined and progressive length and/or diameter increments along the body 602 to assist the clinician in determining a depth and/or diameter of the resulting tract or passageway formed within the patient's body. In addition, the dilator body 602 may feature a hydrophilic outer coating over an entirety of its length for added lubricity to more easily slip through subcutaneous tissues.

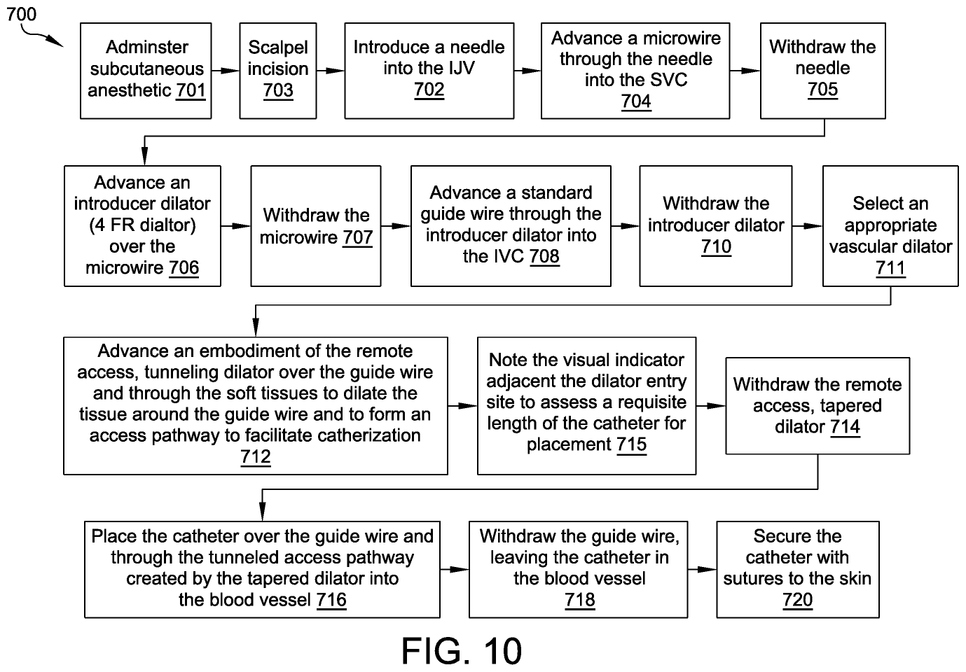

A standard micropuncture kit, along with embodiments of the remote access, tapered tunneling dilator 100, 200, 300, 400, 500, 600, discussed above in relation to FIGS. 3-9, may enable a method of remotely placing a catheter (e.g., a CVC, a dialysis catheter, a larger long-term catheter) via a jugular vein such as the IJV 50 from an approach that is posterior to the SCM 52 (FIGS. 2A-2B). FIG. 10 provides a flowchart depicting an exemplary method of using embodiments of the remote access, tapered tunneling dilator 100, 200, 300, 400, 500, 600 to remotely place a catheter. In one embodiment, the method (700) may initiate with subcutaneously administering a local anesthetic (701) before making a small scalpel incision (703) and puncturing the IJV 50 from a remote infraclavicular access point posterior to the SCM 52 of the neck, using a straight or pre-bent micropuncture or introducer needle (e.g., a 21 g needle) and ultrasonic guidance (702). Once introduction of the tip of the needle is confirmed by ultrasound to be within the IJV 50, the clinician may advance a microwire (e.g., a 0.018 inch wire) through the needle and into the SVC 58 (704) before withdrawing the needle (705). The clinician may then advance an introducer dilator (e.g., a 4 FR diameter dilator) from the micropuncture kit over the microwire (706) before withdrawing the microwire (707). Next, a standard guide wire (e.g., 0.035 inch guide wire, 0.038 inch guide wire) may be advanced through the introducer dilator (708) into the inferior vena cava (IVC) 60 and the introducer dilator may be withdrawn (710) prior to beginning the process of vascular dilation.

Before dilation begins, the clinician may select an appropriate tapered vascular dilator 100, 200, 300, 400, 500, 600 (711) from a selection of vascular dilators of differing configurations, where the selected vascular dilator may feature a length, tapered diameter range, taper slope, and/or variable material hardness most suitable to the patient's size, a target location within the patient's body, and/or other factors. Then, holding the guide wire, an embodiment of the remote access, tunneling dilator 100, 200, 300, 400, 500, 600 may be advanced over the guide wire and through the soft tissues surrounding or confronting the guide wire, thereby using a single dilator to dilate the tissues around the guide wire, thereby forming an access pathway to facilitate catheterization (712) in diameters of 6 FR to 24 FR in a single dilation step.

Once dilation is complete, the dilator 100, 200, 300, 400, 500, 600 may be withdrawn (714). Before, during, or after the tapered vascular dilator is withdrawn (714), the clinician may note the visual indicator 120, 220, 320, 420, 520, 620 adjacent the entry site to assess a requisite length of any catheter for placement (715), and the CVC, dialysis catheter, or other long term catheter may be placed over the guide wire and advanced through the tunneled access pathway created by the dilator 100, 200, 300, 400, 500, 600 into the blood vessel (716). The guide wire may then be withdrawn, leaving the CVC in the blood vessel (718), and the catheter may be secured with sutures to the skin (720). While the method (700) describes placing a catheter from a posterior approach through the IJV 50, to the SVC 58, and to the IVC 60, similar dilation techniques using embodiments of the remote access, tapered tunneling dilator 100, 200, 300, 400, 500, 600 may be used for catheter placement via any appropriate approach through the vascular system.

Using existing dilator systems, a plurality of increasingly sized dilators, including 6 FR, 8 FR, 10 FR, 12 FR, and 14 FR dilators, must be progressively advanced over the guide wire and removed to achieve the ultimate 14 FR pathway necessary to place the CVC, dialysis catheter, or other long-term catheter. Further, when placing an AngioVac device for percutaneous removal of undesirable materials from the intervascular system, traditional dilation would continue beyond 14 FR by progressively placing and removing 16 FR, 18 FR, 20 FR, 22 FR, and finally 24 FR dilators before introducing the AngioVac. Using an embodiment of the remote access, tunneling dilator 100, 200, 300, 400, 500, 600, along with an embodiment of the exemplary dilation method (700), discussed above, the steps of placing and removing numerous progressive dilators are replaced with the placement and removal of a single tapered dilator, resulting in a dilation process that requires less time, expense, and bodily stress and damage than traditional devices and methods.

In one embodiment, the disclosed dilator may be provided as part of a kit including the remote access, tunneling dilator 100, 200, 300, 400, 500, 600 and the micropuncture system discussed above, including the straight or pre-bent needle, the syringe, the 4 FR dilator, the guide wire, and the catheter.

The dilator and methods of use described above may be used for single stick, tunneled CVC placement into any appropriate vein from any appropriate approach. Embodiments may be particularly advantageous for remote access via the IJV from an approach that is posterior to the SCM. Use of embodiments of the remote access, soft tissue tunneling dilator and associated methods discussed above provides a number of benefits over existing CVC placement mechanisms, including: (1) Providing for patient choice: Patients given a choice of the standard two-stick system or the Single stick system unanimously choose the single stick. A single stick, posterior approach allows the patient to more freely turn his or her head without pain, and any residual cosmetic scar is not on the patient's neck; (2) More accessibility: Emergency room personnel may have placed a cervical collar on the patient so traditional two-stick access is not available. Additionally, the traditional two-stick site may not be available because of skin burns or skin damaged by radiation therapy or lymphadenopathy; (3) Fewer Patient Complications: Patients do not have to hold their breath during the single-stick procedure using embodiments of the disclosed dilator. Many patients requiring catheters are in respiratory distress. The lateral/posterior approach avoids causing pneumothorax and cardiac tamponade. Further, a one-stick procedure reduces the chance of air embolism that happens occasionally with the two-stick procedure. There is also less bleeding because the tapered configuration of the remote access, tunneling dilator eliminates the need to exchange progressively sized dilators to accommodate larger catheters, and there is less risk of infection because access to the vein is further away from the lateral/posterior puncture site. It also eliminates the need for peel away sheaths. In addition, access using the disclosed remote access, tunneling dilator 100, 200, 300, 400, 500, 600 requires less time to perform the procedure because it is accomplished using a single tapered dilator, rather than numerous exchanged dilators that increase in diameter, because dilation may occur without the time and expense of an angioplasty balloon catheter for advancement through vascular strictures, and because the visual indicators disposed upon the dilator allow for easy measurement/assessment of the catheter length necessary to reach the target point within the body.

II. Soft Tissue Dilation

In addition to the placement of catheters into the vascular system, as described above, embodiments of the remote access, soft tissue tunneling dilator may be implemented in a variety of percutaneous/subcutaneous tunneling procedures including, for example, accessing the abdominal cavity for placement of a peritoneal drainage/dialysis catheter, accessing the chest cavity for chest tube placements, for placement of gastrostomy feeding tubes, for placement of paracentesis catheters, and/or for accessing the kidney(s) for providing approach by a variety of urological instruments. Rather than advancing multiple progressively larger dilators through the body, the disclosed dilators are useful in establishing remote access due to their ability to safely and effectively navigate subcutaneous features in the body, and to dilate internal passageways to achieve large subcutaneous tracts with a single dilator, enabling dilation procedures to be accomplished more quickly, at less expense, and for the patient to experience significantly less pain.

a. Fascial Dilation

Fascial dilators are used in many medical procedures, ranging from simple vascular access to major cardiac surgery, and by many physicians across all specialties. Exemplary procedures include vascular dilation to allow various therapies and soft tissue dilation to allow access to internal organs or vessels, including but not limited to gastrostomy tubes, abscess drains, peritoneal or pleural drains, chest tube placements, paracentesis catheters, and hemodialysis catheters. Currently such fascial dilation involves utilizing multiple dilators progressing in size until the final tract is large enough for the permanent or usable catheter. This process requires multiple steps to establish the correct size tract. Embodiments of the continuously tapered fascial dilator discussed herein will allow these techniques and procedures to be performed in a single step with a single dilator, saving time and money as well as reducing complications.

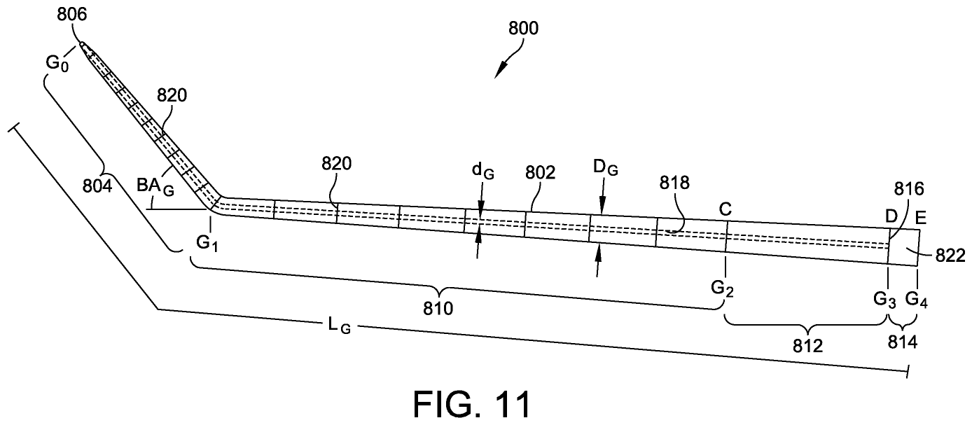

FIG. 11 illustrates a side view of one embodiment of a remote access, tunneling, continuously tapered fascial dilator 800. As shown in FIG. 11 and in one embodiment, the fascial dilator 800 may have a similar configuration to the dilators 100-600 discussed above, with differing sections, lengths, taper dimensions, and material hardness variations. In this embodiment, the curved fascial dilator 800 may have a total length, L_{G}, of 41 cm separated into four zones extending between Markers G₀-G₁, G₁-G₂, G₂-G₃, and G₃-G₄, including a dilator body 802 having a distal end section 804 extending 9 cm from a tapered distal tip 806 at Marker G₀ to Marker G₁, a straight tapered section 810 extending 24 cm between Markers G₁-G₂, and a proximal body section 812 extending 7 cm between Markers G₂-G₃. The dilator 800 further includes a connector end section 814 disposed adjacent to a proximal end 816 of the dilator body 802, extending 1 cm from Marker G₃ to a terminal proximal end of the dilator 800 at Marker G₄. The connector end section 814 may comprise a 1 cm hub 822 such as, for example, a standard female tapered Luer lock hub to allow attachment of other Luer fitting devices to the dilator 800 for the injection of contrast dye.

In this embodiment, the body 802 of the dilator 800 may form a permanent curve between the distal end section 804 extending between Markers G₀-G₁ and the straight tapered section 810 extending between Markers G₁-G₂. In this embodiment, the permanent curve may form a body angle, BA_{G}, between 30-80 degrees relative to the straight tapered section 810. Notably, in some embodiments, the permanent curve or body angle, BA_{G}, may form any appropriate angle including for example, an angle of 5 degrees, 45 degrees, or 90 degrees, depending on the intended fascial dilation application.

In addition to the permanent curve or body angle, BA_{G}, the body 802 of the dilator 800 may feature a tapered configuration having a tapered outer diameter, D_{G}, that progresses in diameter size distally-to-proximally from the distal tip 806 to the proximal end 816 of the body 802, beginning, in this embodiment, with an outer diameter of 5 FR at the distal tip 806 at the Marker G₀ and increasing by 1 FR every 1 cm through the distal end section 804, from 5 FR to 14 FR between Markers G₀-G₁, then progressively increasing by 1 FR every 3 cm through the straight tapered section 810 from 14 FR to 22 FR between Markers G₁-G₂, and reaching and maintaining a maximum diameter of 22 FR at the proximal body section 812 extending between Markers G₂-G₃. In some embodiments, the distal tip 806 of the dilator body 802 may additionally be tapered by approximately 5 degrees, in a manner that renders the tip rounded rather than blunt, for additional ease of insertion and manipulation.

In one embodiment, a longitudinal through hole 818 may extend through an entirety of the length of the dilator 800. The longitudinal through hole 818 may have an inner diameter, d_{G}, configured to accommodate a standard guide wire (e.g., 0.038 inch diameter) (not shown). In addition, the dilator body 802 may feature a hydrophilic outer coating over an entirety of its length for added lubricity to more easily slip through subcutaneous tissues.

Visual indicators 820 may be disposed upon the body 802 of the dilator 800 at each increasing diameter progression to enable a clinician to determine the outer diameter size entering the body, and, in turn, the size/width of the remote access pathway that will be created by the dilator 800. Additionally or alternatively, the visual indicators 820 may be disposed on the body at defined length increments along the total length, L_{G}, of the dilator 800, enabling the clinician to quickly determine the length of CVC necessary for the particular application based upon the visual indicator 820 aligned with an insertion point the patient's skin when the distal tip 806 of the dilator 800 reaches its subcutaneous target within the patient's body.

In one embodiment, the body 802 of the remote access, tunneling fascial dilator 800 may also feature a variable material hardness along the length of the body, L_{G}. For example, in this embodiment, the body 802 may have a material hardness or Shore durometer rating of 80D-90D within the distal end section 804 where the diameter ranges from 7 FR to 12 FR, while the remainder of the dilator body 802 may be 65D. This variation enables the dilator to tunnel through tougher tissue oftentimes encountered by fascial dilators. Additional embodiments of the dilator 800 may vary in section length, taper, and material hardness as desired and/or appropriate.

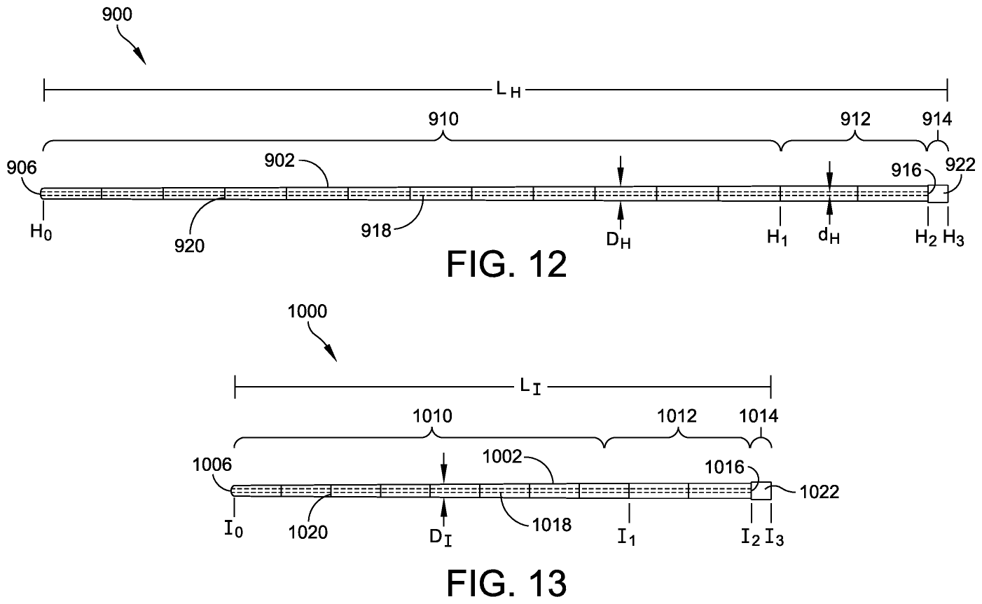

FIGS. 12-13 illustrate side views of two additional straight, remote access, continuously tapered fascial dilators, each having a different overall length and taper angle. Specifically, FIG. 12 illustrates a side view of one embodiment of a remote access, straight tunneling dilator 900 for use in fascial dilation applications. In this embodiment, the straight dilator 900 is shown broken down into 3 zones extending between Markers H₀-H₁, H₁-H₂, and H₂-H₃ for a total length, L_{H}, of 39 cm, including a dilator body 902 having a tapered section 910 extending 32 cm from a rounded distal tip 906 at Marker H₀ to Marker H₁ and a proximal body section 912 extending 6 cm between Markers H₁-H₂. The dilator 900 further includes a connector end section 914 disposed adjacent to a proximal end 916 of the dilator body 902, extending 1 cm from Marker H₂ to a terminal proximal end of the dilator 900 at Marker H₃. The connector end section 914 of the dilator 900 may comprise a hub 922 such as, for example, a standard female tapered Luer lock hub to allow attachment of other Luer fitting devices to the dilator 900 for the purpose of, for example, injecting contrast dye.

In one embodiment, a longitudinal through hole 918 may extend through an entirety of the length of the dilator 900. The longitudinal through hole 918 may have an inner diameter, d_{H}, configured to accommodate a standard guide wire (e.g., 0.038 inch diameter) (not shown). In addition, the dilator body 902 may include a hydrophilic outer coating over an entirety of its length for added lubricity to more easily slip through subcutaneous tissues.

In this embodiment, the body 902 of the dilator 900 may feature a tapered configuration having a tapered outer diameter, D_{H}, that progresses in diameter size distally-to-proximally from the distal tip 906 to the proximal end 916 of the body 902, beginning, in this embodiment, with an outer diameter of 6 FR at the distal tip 906, then progressively increasing through the straight tapered section 910 by 1 FR every 2 cm, from 6 FR to 22 FR, between Markers H₀-H₁, and reaching and maintaining a maximum diameter of 22 FR at the non-tapered proximal body section 912 extending between Markers H₁-H₂, as detailed in FIG. 12.

Visual indicators 920 may be disposed upon the body 902 of the dilator 900 at each increasing diameter progression to enable a clinician to determine the outer diameter size entering the body, and, in turn, the size of the remote access pathway that will be created by the dilator 900. Additionally or alternatively, the visual indicators 920 may be disposed on the body at defined length increments along the total length, L_{H}, of the dilator 900, enabling the clinician to quickly determine the length of catheter necessary for the particular application based upon the visual indicator 920 aligned at the entry point when the distal tip 906 of the dilator 900 reaches its subcutaneous target within the patient's body.

FIG. 13 illustrates a side view of one embodiment of another exemplary straight, remote access, facial dilator 1000. Dilator 1000 may be similar in configuration to dilator 900 and includes the same sections and components, discussed above, with varying section lengths and taper slopes to meet the needs of different fascial dilation applications (e.g., pathway lengths, patient sizes, tissue types to be dilated). More specifically and in one embodiment, dilator 1000 of FIG. 13 may have a total length, L_{I}, of 22 cm extending between a rounded or angled distal tip 1006 and a proximal end of a hub 1022. A body 1002 of the dilator 1000 may feature a tapered configuration having a tapered outer diameter, D_{I}, that progresses in diameter size distally-to-proximally from the distal tip 1006 to a proximal end 1016 of the body 1002, beginning, in this embodiment, with an outer diameter of 6 FR at the distal tip 1006 and progressively increasing through a 16 cm straight tapered section 1010 by 1 FR every 1 cm, from 6 FR to 22 FR, between Markers I₀-I₁, reaching and maintaining a maximum diameter of 22 FR through a 5 cm non-tapered proximal body section 1012 extending between Markers I₁-I₂. The dilator 1000 further includes a connector end section 1014 disposed adjacent to the proximal end 1016 of the dilator body 1002, extending 1 cm from Marker I₂ to a terminal proximal end of the dilator 1000 at Marker I₃. The connector end section 1014 may comprise a hub 1022 such as, for example, a standard female tapered Luer lock hub to allow attachment of other Luer fitting devices to the dilator 1000 for the purpose of, for example, injecting contrast dye.

The dilator 1000 may include a through hole 1018 configured to accommodate a standard guide wire (e.g., 0.038 inch) and may include sets of visual indicators 1020 disposed at predetermined and progressive length and/or diameter increments along the body 1002 to assist the clinician in determining a depth and/or diameter of the resulting tract or passageway formed within the patient's body. In addition, the dilator body 1002 may feature a hydrophilic outer coating over an entirety of its length for added lubricity to more easily slip through subcutaneous tissues.

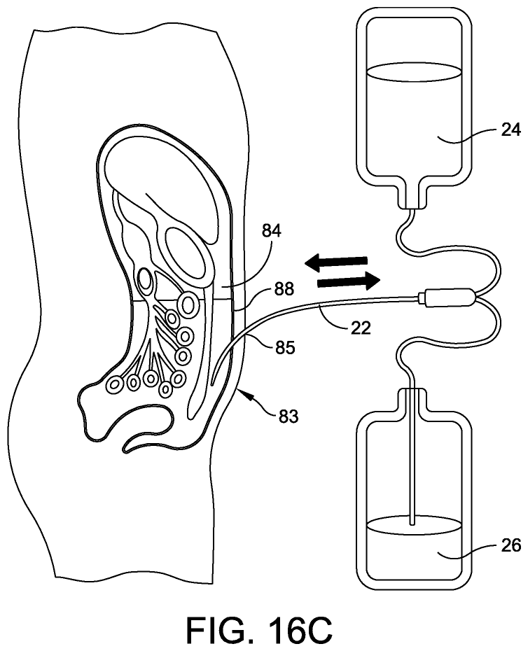

To illustrate an exemplary use of embodiments of the tapered fascial dilator 800, 900, 1000, discussed above, FIGS. 16A-16B show respective anterior and lateral cross-sectional views of a human peritoneal cavity 84 having a pelvic abscess 86, while FIG. 16C illustrates a peritoneal catheter 22, as placed into the peritoneal cavity 84 for peritoneal drainage and dialysis of the pelvic abscess 86. During peritoneal dialysis, a cleansing fluid 24 (e.g., dialysate) is circulated through the peritoneal catheter 22 inside the peritoneal (i.e., abdominal) cavity 84. The cleansing fluid 24 absorbs waste products from blood vessels in the peritoneum (i.e., the abdominal lining) 88 and is then is drawn back out of the body and discarded as waste 26, as shown in FIG. 16C.

The success of peritoneal dialysis as renal replacement therapy hinges upon the patient possessing functional peritoneal access, which is obtained using the catheter 22 that bridges the abdominal wall and serves as a controlled cutaneoperitoneal fistula. Because patients come in all sizes and shapes with a variety of medical conditions, different types of commercially-available peritoneal catheters having a variety of alternative design features are utilized (e.g., Tenckhoff catheters with a coiled-tip, two cuffs, and a straight or swan neck intercuff segment; Tenckhoff catheters with a straight-tip, two-cuffs, and a straight or swan neck intercuff segment; an extended catheter with a coiled-tip and one-cuff; a two-cuff extension catheter with a swan neck intercuff segment and a titanium connector; a straight-tip catheter with silicone disks; a straight-tip catheter with a tungsten weight; and a Dracon flange and a silicone bead below and adjoining a deep cuff).

The type of peritoneal catheter 22 selected and/or the placement of the peritoneal catheter 22 varies based upon many factors including, for example, where the patient's beltline is, abdomen size (e.g., large and/or rotund), severe obesity, drooping skin folds, intestinal stomas, feeding tubes, urinary or fecal incontinence, yeast intertrigo, and/or bathing habits (e.g., a desire to take deep baths). After selection of an appropriate catheter type, catheter size, and an access (i.e., entry/exit) site to suit the patient's particular needs, the peritoneal catheter 22 may be placed in an operating room or at the patient's bedside utilizing an embodiment of the tapered fascial dilator 800, 900, 1000. According to best practices, the subcutaneous tunnel, tract, or pathway formed by the fascial dilator 800, 900, 1000 should not have a diameter that exceeds the diameter of the selected catheter 22, and the access site should form the smallest diameter hole in the skin that allows passage of the peritoneal catheter 22. Using an embodiment of the tapered fascial catheter 800, 900, 1000, such a tract may be formed using a single dilator that forms a more precise tunnel, helping to achieve these best practices while saving procedure time and equipment expense.

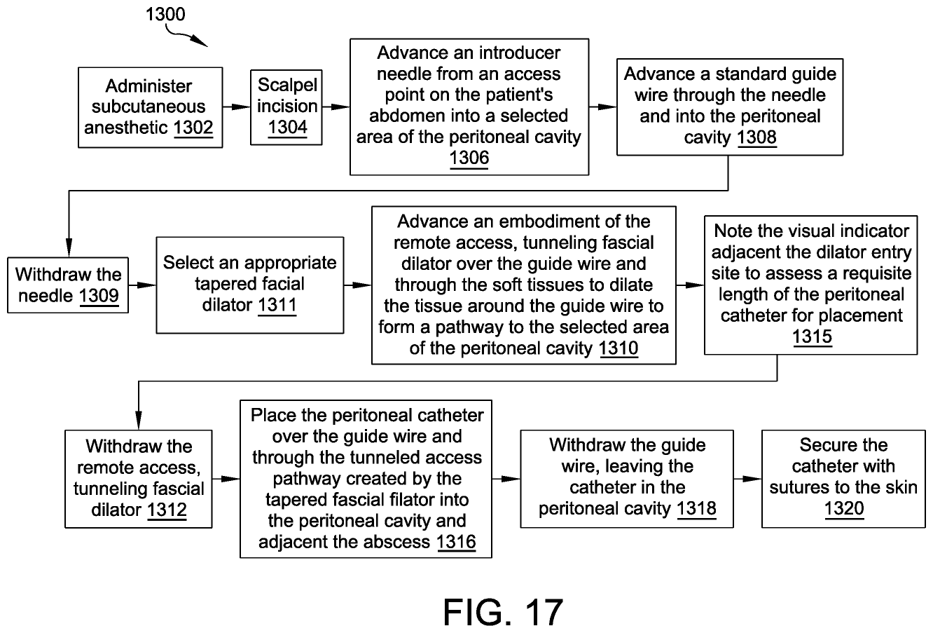

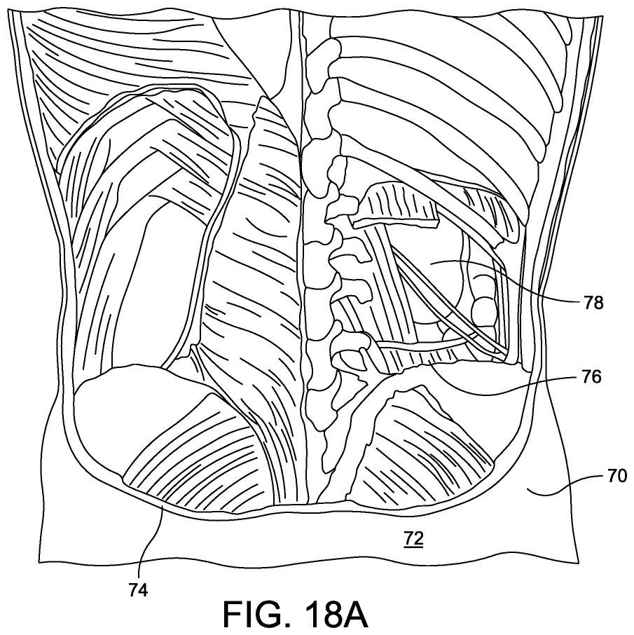

FIG. 17 provides a flowchart depicting the steps of an exemplary method (1300) of using an embodiment of the continuously tapered fascial dilator 800, 900, 1000, discussed above, for dilating a subcutaneous access pathway to a target area within a patient's body for, in this embodiment, placement of a peritoneal catheter 22 for peritoneal drainage and dialysis of a pelvic abscess 86, as shown in FIGS. 16A-16C. In one embodiment, the method (1300) may initiate with subcutaneously administering a local anesthetic (1302) before making a small scalpel incision (1304) at an access point 85 and advancing an introducer needle from the selected access point 85 on the patient's abdomen 83, opposite the peritoneal cavity 84 (FIG. 16C), and into a selected area of the peritoneal cavity 84 (FIG. 16A-16C) (1306). In this embodiment, the advancement of the introducer needle may be made using ultrasonic guidance. Once introduction of the tip of the needle is confirmed by ultrasound to be within the selected area of the peritoneal cavity 84, the clinician may advance a standard guidewire (e.g., a 0.038 inch guide wire) through the needle and into the peritoneal cavity 84 (FIG. 16A-16C) (1408) prior to withdrawing the needle (1309) and beginning the process of fascial dilation.

Before dilation begins, the clinician may select an appropriate tapered facial dilator 800, 900, 1000 (1311) having the length, tapered diameter range, taper slope, and/or variable material hardness most suitable to the type, thickness, and/or density of the tissues to be traversed (e.g., skin, fat, muscle), the patient's size, and/or the selected target area of the patient's body to be accessed. Then, holding the guide wire, an embodiment of the selected remote access, tapered fascial dilator 800, 900, 1000 may be advanced over the guide wire and through the soft tissues including the skin 72, fat 74, muscle 76, and other overlying tissues (FIG. 16A-16B), thereby using a single fascial dilator and not more than one dilation step to dilate the tissue around the guide wire to form an access pathway of a desired diameter from the access point 85 on the patient's abdomen 83 to the selected area within the peritoneal cavity 84 (1310).

Once dilation is complete, the tapered fascial dilator 800, 900, 1000 may be withdrawn (1312). Before, during, or after the tapered fascial dilator is withdrawn (1312), the clinician may note the visual indicator 820, 920, 1020 adjacent the access point 85 to assess a requisite length of any peritoneal catheter 22 for placement (1315), and the peritoneal catheter 22 may be placed over the guide wire and advanced through the tunneled access pathway created by the dilator 800, 900, 1000 into the peritoneal cavity 84 and, in turn, the pelvic abscess 86 (1316). The guide wire may then be withdrawn, leaving the catheter 22 in the peritoneal cavity 84 (1318), and the catheter may be secured with sutures to the skin 72 (1320).

Using existing dilator systems, a plurality of increasingly sized fascial dilators, ranging in diameter from 6 FR to 22 FR, must be progressively advanced over the guide wire and removed to achieve a pathway diameter of 22 FR that is oftentimes necessary to place the peritoneal catheter 22. Using an embodiment of the remote access, tunneling fascial dilator 800, 900, 1000, along with the exemplary dilation method (1300), discussed above, the steps of placing and removing numerous progressive dilators are replaced with the placement and removal of a single dilator via a single dilation step, resulting in a dilation process that requires less time, expense, and bodily stress and damage than traditional devices and methods.

b. Renal Access Dilation

Chronic kidney disease is a worldwide public health problem, a social calamity, and an economic catastrophe. In 2010, it was estimated that 6 million people worldwide would need renal replacement therapy costing approximately 28 Billion dollars. Urologists, nephrologists, and interventional radiologists have continually strived to bring these numbers down. First described in 1955 by urologist Dr. Willard Goodwin et. al. as a minimally invasive treatment for urinary obstruction causing marked hydronephrosis, percutaneous nephrostomy placement or access quickly found use in a wide variety of clinical indications in both dilated and non-dilated systems. There now are many indications for percutaneous access to the kidneys including infections, trauma, calculi including staghorn and ureteral types, and neoplasms.

There have been many different approaches to dilating a tract to the kidneys through the patient's flank for placement of tubes into the collecting system of the kidneys for drainage purposes or for access for percutaneous kidney stone removal of large stones. These approaches include serially introduced progressive sizes of renal access dilators, Amplatz dilator sets, and metal coaxial dilators. Currently, renal access pathways are preferably formed using a nephrostomy balloon dilator designed for ease of use, one step dilation with minimized operative time, and decreased fluoroscopic time with decreased risk of hemorrhage. However, in up to 25% of patients, the nephrostomy balloon fails to work. Causes of these failures include prior renal surgery, stone burden, patient BMI, and history of pyelonephritis. Beyond the unacceptable failure rate, nephrostomy balloons are associated with high cost and have a fixed length. When the balloon fails or its use is inappropriate, legacy methods of serial dilation must be performed, involving advancing into the body a progressive series of up to 10 dilators varying in diameter from 8 FR to 30 FR. Such large tracts are necessary for urological instruments to be placed into the kidney for nephrostolithotomy and nephroscopy.

Use of a single continuously tapered renal access dilator to replace the series of progressively larger dilators offers the advantage of having one dilation step and one dilator, thereby reducing procedural time and potential complications. In this regard, many of the advantages of the nephrostomy balloon are preserved, but at a much lower cost. Savings in operative time and decreased radiation under fluoroscopy are similarly achieved, rendering embodiments of the continuously tapered renal access dilator described herein particularly attractive in developing nations where the economics of dialysis render it infeasible for the increasing population of patients needing renal replacement therapy.

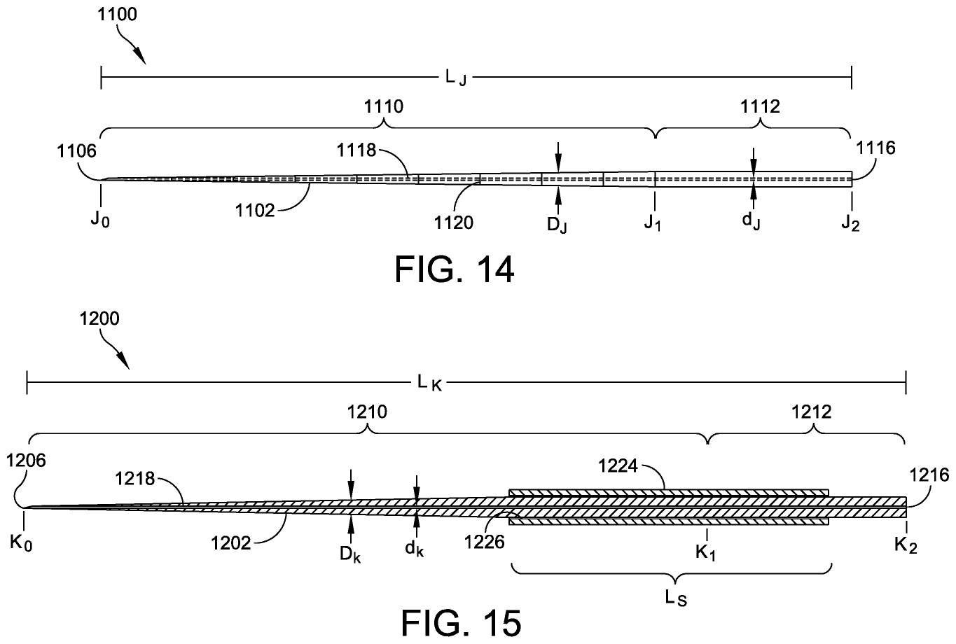

FIG. 14 illustrates a side view of one embodiment of a remote access, tapered renal access dilator 1100 for use in achieving pathways of varying sizes (e.g., from 6 FR up to 30 FR) for renal access by a variety of urological instruments. In this embodiment, the renal access dilator 1100 is shown broken down into two zones extending between Markers J₀-J₁ and J₁-J₂ for a total length, L_{J}, of 42 cm, including a dilator body 1102 having a tapered section 1110 extending 32 cm from a pointed distal tip 1106 at Marker J₀ to Marker J₁ and a proximal body section 1112 extending 10 cm between Markers J₁-J₂. The dilator 1100 may exclude a connector end section and hub, as a hub is unnecessary without the need to inject contrast dye.

In this embodiment, the body 1102 of the dilator 1100 may feature a tapered configuration having a tapered outer diameter, D_{J}, that progresses in diameter size distally-to-proximally from the distal tip 1106 to the proximal end 1116 of the body 1102, beginning, in this embodiment, with an outer diameter of 6 FR at the distal tip 1106, then progressively increasing through the straight tapered section 1110 by 1 FR every 2 cm, from 6 FR to 22 FR, between Markers J₀-J₁, and reaching and maintaining a maximum diameter of 22 FR through the non-tapered proximal body section 1112 extending between Markers J₁-J₂, as detailed in FIG. 14.

In one embodiment, a longitudinal through hole 1118 may extend through an entirety of the length of the dilator 1100. The longitudinal through hole 1118 may have an inner diameter, d_{J}, configured to accommodate a standard guide wire (e.g., 0.038 inch diameter) (not shown).

Visual indicators 1120 may be disposed at predetermined and progressive length and/or diameter increments along the body 1102 to assist the clinician in determining a depth and/or diameter of the resulting tract or passageway formed within the patient's body. In addition, the dilator body 1102 may feature a hydrophilic outer coating over an entirety of its length for added lubricity to more easily slip through subcutaneous tissues.

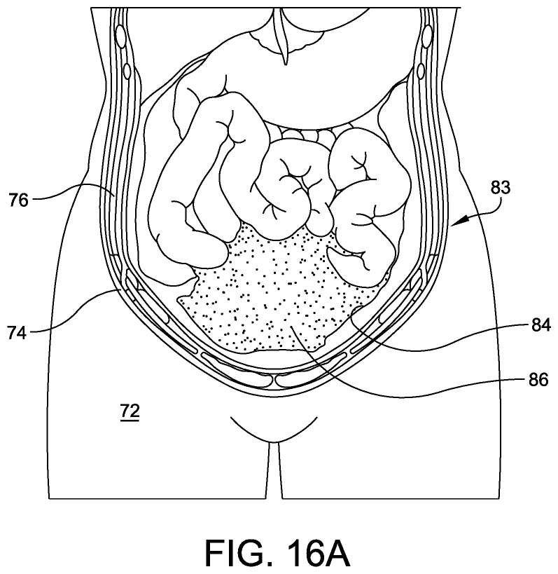

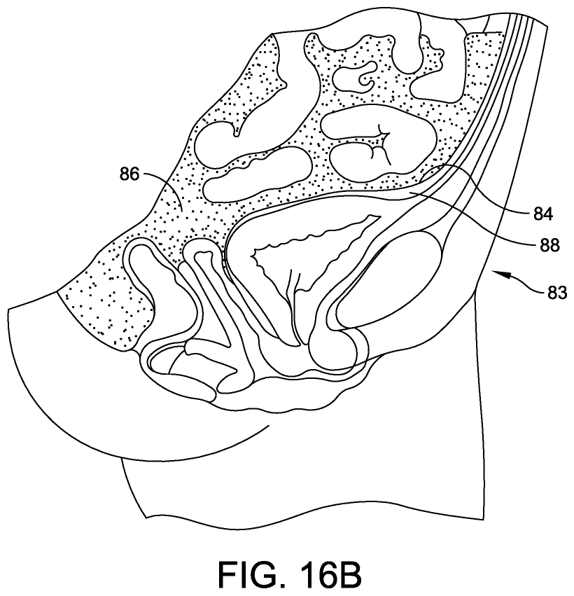

FIG. 15 illustrates a cross-sectional view of an additional exemplary renal access dilator 1200, which is similar in configuration to dilator 1100, discussed above, and includes the same sections and components with varying section lengths and taper slopes to meet the needs of different renal access dilation applications (e.g., pathway lengths, sizes of patients, tissue type). More specifically and in this embodiment, the dilator 1200 may have a total length, LK, of 44 cm extending between a pointed distal tip 1206 and a proximal end 1216. A body 1202 of the dilator 1200 may feature a tapered configuration having a tapered outer diameter, DK, that progresses in diameter size distally-to-proximally from the distal tip 1206 to the proximal end 1216 of the body 1202, beginning, in this embodiment, with an outer diameter of 6 FR at the distal tip 1206 and progressively increasing through a 24 cm straight tapered section 1210 by 1 FR every 1 cm, from 6 FR to 30 FR, between Markers K₀-K₁, reaching and maintaining a maximum diameter of 30 FR through a 20 cm non-tapered proximal body section 1212 extending between Markers K₁-K₂.

The dilator 1200 may include a through hole 1218 configured to accommodate a standard guide wire (e.g., 0.038 inch) and may include sets of visual indicators (not shown) disposed at predetermined and progressive length and/or diameter increments along the body 1202 to assist the clinician in determining a depth and/or diameter of the resulting tract or passageway formed within the patient's body. In addition, the dilator body 1202 may feature a hydrophilic outer coating over an entirety of its length for added lubricity to more easily slip through subcutaneous tissues.

In some embodiments, the renal access dilator 1200 may be used in conjunction with a sheath 1224, as shown in FIG. 15. Once the subcutaneous tract is dilated to the desired size, the sheath 1224 may be inserted into the tract over the dilator to maintain the tract opening before the dilator 1200 is removed. In this embodiment, the sheath may have a length, L_{S}, of 16 cm and include a through hole 1226 having a diameter configured to slide over the maximum diameter of the dilator 1200.

In some embodiments, the dimensions and the taper slopes of the straight tapered section 1210 and the proximal body section 1212 of the dilator 1200 may vary as appropriate for differing, namely smaller or shorter, intended tract sizes and lengths. For example, the straight tapered section 1210 may progressively increase through 18, 20, or 22 cm by 1 FR every 1 cm, from 6 FR to 24 FR, from 6 FR to 26 FR, and from 6 FR to 28 FR, respectively, between Markers K₀-K₁. The 18, 20, or 22 cm straight tapered section 1210 may combine with the 20 cm proximal body section 1212 for a total length of 38, 40, or 42 cm. Embodiments of the sheath 1224 may, in turn, be configured to slide about or over the respective maximum diameter of the dilator 1200. In this regard, embodiments of the renal access dilator 1200 may provide a dilated renal access tract or pathway having an extremely large diameter range beginning a 6 FR and increasing to 24 FR, 26 FR, 28 FR, and finally 30 FR using a single dilator.

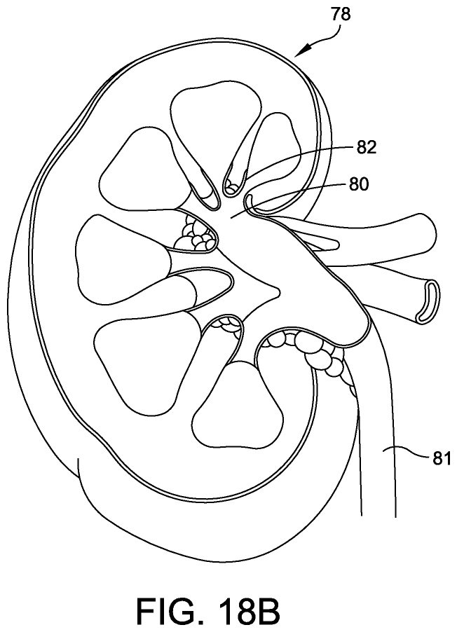

A standard micropuncture kit, along with embodiments of the remote access, tunneling renal access dilator 1100, 1200, discussed above in relation to FIGS. 14-15, may enable a method of forming a subcutaneous pathway through a patient's posterior flank 70, through layers of skin 72, fat 74, muscle 76, and other overlying tissues, and into the patient's kidney 78, shown in FIGS. 18A-18B, where an embodiment of a sheath, such as the sheath 1224 discussed above in relation to FIG. 15, may be introduced into a major calyx 80 of the kidney 78, a minor calyx 82 of the kidney 78, or another selected target area of the kidney 78 to facilitate access by one or more urology instruments.

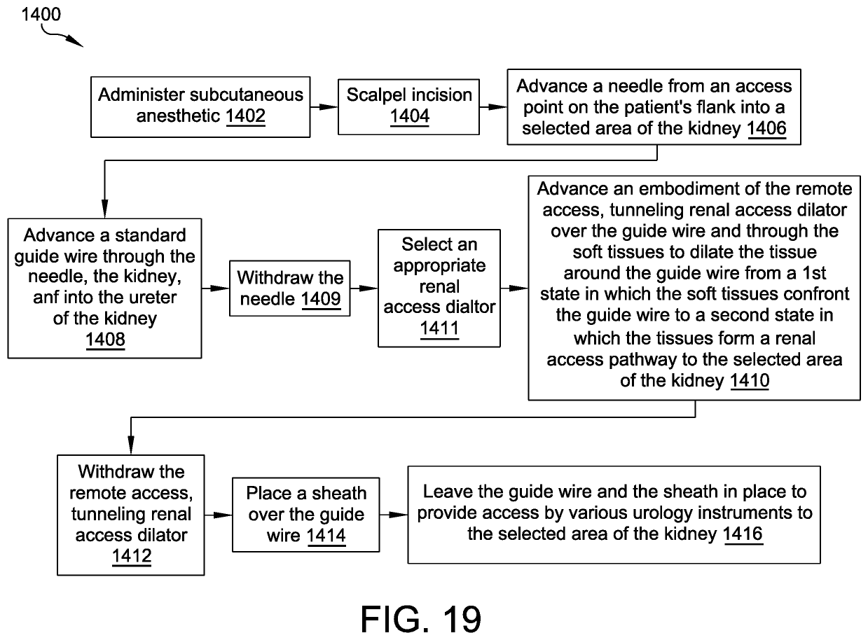

FIG. 19 provides a flowchart depicting an exemplary renal access dilation method (1400) using embodiments of the remote access, tapered renal access dilator 1100, 1200. In one embodiment, the method (1400) may initiate with subcutaneously administering a local anesthetic (1402) before making a small scalpel incision (1404) and advancing an introducer needle from an access point on the patient's posterior flank 70 (FIG. 18A) into a selected area of the kidney 78 (FIG. 18B) (1406). In this embodiment, the advancement of the introducer needle may be made using ultrasonic guidance. Once introduction of the tip of the needle is confirmed by ultrasound to be within the selected area of the kidney 78, the clinician may advance a standard guidewire (e.g., a 0.038 inch guide wire) through the needle, the kidney 78, and, for example, into the ureter 80 of the kidney 78 (FIG. 18B) (1408) prior to removing the needle (1409) and beginning the process of renal access dilation.

Before dilation begins, the clinician may select an appropriate tapered renal access dilator 1100, 1200 (1411) having the length, tapered diameter range and slope, and/or variable material hardness values most suitable to the type, thickness, and/or density of tissues to be traversed (e.g., skin, fat, muscle), the patient's size, and/or the selected target area of the kidney 78 to be accessed. Then, holding the guide wire, the embodiment of the selected remote access, tapered, renal access dilator 1100, 1200 may be advanced over the guide wire and through the soft tissues including the skin 72, fat 74, muscle 76, and other overlying tissues (FIG. 18A), thereby using a single renal access dilator, and one dilation step, to dilate the soft tissues around the guide wire from a first state in which the tissues confront the guide wire to a second state in which the tissues form a renal access pathway of a desired diameter from the patient's flank 70 to the selected target area of the kidney 78 (1410).

Once dilation is complete, the renal access dilator 1100, 1200 may be withdrawn (1412), and a select and corresponding size of a sheath, such as an embodiment of the sheath 1224 of FIG. 15, may be placed over the guide wire (1414), leaving the guide wire and the sheath in place such that various urology instruments may be placed through the sheath to access the selected area of the kidney 78 (1416) via the renal access pathway.

As discussed above, existing renal access dilator systems require a plurality of increasingly sized dilators, including a range of 10 FR to 30 FR dilators, to be progressively advanced over the guide wire and removed to form an access pathway for a 24 FR, 26 FR, 28 FR, or 30 FR catheter-and-sheath combination, which is placed over the guide wire before the catheter is removed, leaving the guide wire and the sheath in place such that urology instruments may be placed through the sheath to access the selected area of the kidney 78. Using an embodiment of the remote access, tunneling renal access dilator 1100, 1200, along with an embodiment of the exemplary renal access dilation method (1400), discussed above, the steps of placing and removing numerous progressive dilators, as well as the step of placing and removing a catheter-and-sheath combination, are replaced with the advancement and removal of a single tapered, renal access dilator that forms a pathway into which the sheath may be directly placed. Thus, embodiments of the disclosed tapered renal dilator 1100, 1200 and the method (1400) enable a renal access dilation process that requires less time, expense, and bodily stress and damage than traditional devices and methods.

Embodiments of the remote access, tunneling dilator 100-1200 and methods of use discussed herein provide exemplary configurations only. Additional dilator embodiments may have any appropriate length, diameter, curvature, and/or material hardness variance, and may include any appropriate degree or slope of tapering, or any appropriate progressive stepping in outer diameter French catheter sizes. For example and as described above, dilator embodiments may feature a long length with only a few increasing increments in French catheter size for access through the left IJV 50. Other embodiments of the dilator may feature a shorter length with only a few increasing increments in French catheter size for the right IJV. Still other embodiments may feature a thinner body made for pediatric patients. Other embodiments may be configured in length, taper, and maximum diameter for soft tissue applications such as fascial and renal access applications to achieve a very large tract size with a single dilator, as discussed above. Embodiments of the dilator and/or the accompanying pre-bent or straight needle may feature ultra-visible tips for better visualization under ultrasound guidance.

Embodiments of the remote access, tunneling dilator 100-1200 may be formed of any appropriate material to achieve the desired configurations and stiffnesses. Some embodiments may be formed of a barium sulfate loaded polyurethane.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A method of dilating a vascular system for remotely placing a catheter via a jugular vein of a patient, the method comprising:
providing a tapered vascular dilator having a tapered body extending from a proximal end to a distal tip and having a proximal body section and a straight tapered section disposed proximally-to-distally therebetween, wherein:
the straight tapered section extends along a straight tapered length having an outer diameter that slopes distally-to-proximally from a minimum distal diameter to a maximum proximal diameter; and
the proximal body section extends along a proximal body length having the maximum proximal diameter;
using an introducer needle, puncturing an internal jugular vein (IJV) from a remote access point posterior to a sternocleidomastoid muscle (SCM) of the patient;
advancing, distally-to-proximally, the tapered vascular dilator over a standard guide wire from the remote access point, through a plurality of soft tissues surrounding the guide wire, and into an inferior vena cava (IVC), thereby dilating an access pathway from the remote access point to the IVC in not more than one dilation step, the access pathway having a diameter sized to receive the catheter to facilitate catheterization;
removing the tapered vascular dilator; and
placing the catheter over the guide wire and through the access pathway created by the tapered vascular dilator into the IVC.

2. The method of claim 1, further comprising, after the puncturing the IJV and prior to the advancing the tapered vascular dilator:
advancing a microwire through the introducer needle into a superior vena cava (SVC);
withdrawing the introducer needle;
advancing an introducer dilator over the microwire;
withdrawing the microwire;
advancing the guide wire through the introducer dilator to the IVC; and
withdrawing the introducer dilator.

3. The method of claim 1, wherein the diameter of the access pathway is between 5 FR and 24 FR.

4. The method of claim 1, further comprising, prior to the removing the tapered vascular dilator:
assessing a length of the access pathway, the assessing comprising determining which one of a plurality of visual indicators disposed distally-to-proximally at a plurality of progressively-increasing length increments along the tapered body aligns with the remote access point; and
selecting the catheter based on the length of the access pathway.

5. The method of claim 1, wherein:
the distal tip of the tapered body has a first material hardness and the proximal end section of the tapered body has a second material hardness; and
the second material hardness is greater than the first material hardness.

6. A method of dilating a subcutaneous access pathway to a target area within a body of a patient, comprising:
forming, using not more than one tapered fascial dilator, an access pathway from an access point on a skin surface of the patient to the target area within the body, wherein the forming the access pathway comprises advancing the tapered fascial dilator, distally-to-proximally, over a guide wire from the access point, through a plurality of soft tissues surrounding the guide wire, and into the target area within the body, the access pathway having a diameter sized to receive a medical implement.

7. The method of claim 6, wherein the not more than one tapered fascial dilator comprises a body extending from a proximal end to a distal tip, the body having a tapered outer diameter that increases distally-to-proximally from a minimum diameter at the distal tip to a maximum diameter at the proximal end.

8. The method of claim 7, wherein:
the minimum diameter of the body of the not more than one tapered fascial dilator is 6 FR;
the maximum diameter of the body of the not more than one tapered fascial dilator is 22 FR; and
the diameter of the access pathway is between 6 FR and 22 FR.

9. The method of claim 7, wherein:
the distal tip of the body has a first material hardness and the proximal end of the tapered body has a second material hardness; and
the second material hardness is greater than the first material hardness.

10. The method of claim 6, wherein the body further comprises a plurality of visual indicators disposed upon the body at a plurality of distally-to-proximally increasing length increments, the method further comprising:
determining which one of the plurality of the visual indicators aligns with the access point on the skin surface to assess a length of the access pathway; and
based upon the length of the access pathway, selecting a length of the medical implement.

11. The method of claim 6, wherein the plurality of the soft tissues comprises one or more of a skin layer, a fat layer, and a muscle layer.

12. The method of claim 11, further comprising:
selecting, prior to the forming the access pathway, the not more than one tapered fascial dilator from a plurality of tapered fascial dilators, the selecting based upon at least one of a size of the patient, a diameter of the medical implement, a location of the target area within the body of the patient, a thickness of one or more of the plurality of the soft tissues, and a density of one or more of the plurality of the soft tissues.

13. The method of claim 6, wherein:
the target area within the body is a peritoneal cavity of the patient; and
the medical implement is a peritoneal catheter.

14. A method of forming an access pathway to a target area of a kidney of a patient using a renal access dilator comprising a tapered body extending from a proximal end to a distal tip, the body having a tapered outer diameter that increases distally-to-proximally from a minimum diameter at the distal tip to a maximum diameter at the proximal end, the method comprising:
advancing an introducer needle from an access point on a posterior flank of the patient into the target area of the kidney;
advancing a guide wire through the introducer needle into the target area of the kidney;
advancing the renal access dilator, distally-to-proximally, over the guide wire from the access point, through a plurality of soft tissues surrounding the guide wire, and into the target area of the kidney, thereby dilating the plurality of the soft tissues along the guide wire from a first state in which the plurality of the soft tissues confront the guide wire to a second state in which the plurality of the soft tissues form a renal access pathway having a diameter sized to receive a select urology instrument;
withdrawing the renal access dilator; and
placing a sheath over the guide wire such that the sheath and the guide wire together maintain the renal access pathway for the select urology instrument from the access point into the target area of the kidney.

15. The method of claim 14, wherein the diameter of the access pathway is between 6 FR and 30 FR.

16. The method of claim 15, wherein:
the minimum diameter at the distal tip of the tapered body is 6 FR; and
the maximum diameter at the proximal end of the tapered body is between 22 FR and 30 FR.

17. The method of claim 14, wherein:
the distal tip of the tapered body has a first material hardness and the proximal end of the tapered body has a second material hardness; and
the second material hardness is greater than the first material hardness.

18. The method of claim 14, further comprising:
prior to the advancing the renal access dilator over the guide wire, selecting the renal access dilator from a plurality of renal access dilators, wherein the selected renal access dilator has a minimum-to-maximum diameter range, a taper slope, and a variable material hardness configured for one or more of a size of the patient, a thickness of one or more of the plurality of the soft tissues, a density of one or more of the plurality of the soft tissues, and a location of the target area of the kidney.

19. The method of claim 14, wherein the plurality of the soft tissues comprises a skin layer, a fat layer, and a muscle layer.

20. The method of claim 14, wherein the tapered body of the renal access dilator further comprises a plurality of visual indicators disposed upon the tapered body at a plurality of distally-to-proximally increasing length increments, the method further comprising:
prior to the withdrawing the renal access dilator, determining which one of the plurality of the visual indicators aligns with the access point on the posterior flank to assess a length of the renal access pathway; and
selecting the select urology instrument based on the length of the renal access pathway.
